(19) United States
(12) Patent Application Publication
(10) Pub. No.: US 2016/0270842 A1
(43) Pub. Date: Sep. 22, 2016

(54) ELECTROSURGICAL DEVICE HAVING CONTROLLABLE CURRENT PATHS

(71) Applicant: Ethicon Endo-Surgery, LLC, Guaynabo, PR (US)

(72) Inventors: Geoffrey S. Strobl, Williamsburg, OH (US); Craig T. Davis, Cincinnati, OH (US); Kevin D. Felder, Cincinnati, OH (US); David C. Yates, West Chester, OH (US); Barry C. Worrell, Centerville, OH (US)

(21) Appl. No.: 14/664,421

(22) Filed: Mar. 20, 2015

(51) Int. Cl. A61B 18/14

(52) U.S. Cl. CPC A61B 18/1445; A61B 2018/0063

(57) ABSTRACT

An electrosurgical instrument. The electrosurgical instrument includes an end effector movable between a first position and a second position, a first jaw member which includes first and second electrically conductive members, and a second jaw member which includes a third electrically conductive member. The first jaw member is movable relative to the second jaw member from an open position to a closed position to grasp a tissue positioned between the first and second jaw members. At least one of the first and second jaw members is adapted to connect to an electrosurgical energy source such that electrosurgical energy can be selectively communicated through the tissue positioned between the first and second jaw members to effect a tissue seal. A distance between the first electrically conductive member and the third electrically conductive member is less than a distance between the second electrically conductive member and the third electrically conductive member.

This application discloses, generally and in various embodiments, electrosurgical devices having various components for controlling current paths within the electrosurgical devices.

Conventional electrosurgical devices apply a uniform level of pressure and a uniform level of energy to a grasped tissue regardless of the thickness or composition of the tissue. Due to variations in the thickness and/or composition of different tissues, the jaws of an end effector can experience permanent deflection when subjected to excess stress during the closing/grasping process. Additionally, the application of too much energy to the grasped tissue can cause unwanted damage to the tissue and the application of too little energy can result in an ineffective seal. Furthermore, the application of the same amount of energy to different portions of the tissue can result in a seal which is less than optimal.

While several devices have been made and used, it is believed that no one prior to the inventors has made or used the device described in the appended claims.

### SUMMARY

In one embodiment, an electrosurgical instrument is provided. The electrosurgical instrument comprises an end effector which is movable between a first position and a second position. The end effector comprises a first jaw member and a second jaw member. The first jaw member comprises a first electrically conductive member and a second electrically conductive member which is electrically isolated from the first electrically conductive member. The second jaw member comprises a third electrically conductive member. The first jaw member is movable relative to the second jaw member from an open position to a closed position to grasp a tissue positioned between the first and second jaw members. At least one of the first and second jaw members is adapted to connect to an electrosurgical energy source such that electrosurgical energy can be selectively communicated through the tissue positioned between the first and second jaw members to effect a tissue seal. A distance between the first electrically conductive member and the third electrically conductive member is less than a distance between the second electrically conductive member and the third electrically conductive member.

In another embodiment, at least one of the first, second and third electrically conductive members is an electrode configured to deliver electrosurgical energy to the tissue positioned between the first and second jaw members.

In another embodiment, the electrosurgical instrument is configured to apply a first level of electrosurgical energy to the first electrically conductive member, and a second level of electrosurgical energy to the second electrically conductive member.

In another embodiment, the first level of electrosurgical energy is greater than the second level of electrosurgical energy.

In another embodiment, the electrosurgical energy is one of the following: a radio-frequency energy and a sub-therapeutic radio-frequency energy.

In another embodiment, the first and third electrically conductive members are configured to collectively apply a first closure pressure to the tissue positioned between the first and second jaw members, and the second and third electrically conductive members are configured to collectively apply a second closure pressure to the tissue. The first closure pressure is greater than the second closure pressure.

In another embodiment, the first jaw member further comprises an electrically insulative member positioned between the first and second electrically conductive members.

In another embodiment, the second jaw member further comprises a fourth electrically conductive member electrically isolated from the third electrically conductive member, wherein the distance between the first and third electrically conductive members is less than a distance between the second electrically conductive member and the fourth electrically conductive member.

In another embodiment, the electrosurgical instrument further comprises a first electrically insulative member positioned between the first and second electrically conductive members, and a second electrically insulative member positioned between the third and fourth electrically conducting members.

In another embodiment, each of the first and second jaw members is adapted to connect to an opposite potential of the electrosurgical energy source such that upon activation, the first and second jaw members conduct bipolar energy through the tissue positioned between the first and second jaw members to effect the tissue seal.

In one embodiment, an electrosurgical surgical instrument is provided. The electrosurgical instrument comprises an end effector and a controller. The end effector is movable between a first position and a second position and comprises a first jaw member and a second jaw member. The first jaw member comprises a first body member and a first electrically conductive member electrically isolated from the first jaw body member. The second jaw member comprises a second jaw body member and a second electrically conductive member electrically isolated from the second jaw body member. The first jaw member is movable relative to the second jaw member from an open position to a closed position to grasp a tissue positioned between the first and second jaw members. At least one of the first and second jaw members is adapted to connect to an electrosurgical energy source such that electrosurgical energy can be selectively communicated through the tissue positioned between the first and second jaw members to effect a tissue seal. The controller is configured to selectively electrically couple the second electrically conductive member with the first electrically conductive member, and the second electrically conductive member with the second jaw body member.

In another embodiment, the electrosurgical instrument further comprises a cutting member, and the controller is further configured to selectively electrically couple the second electrically conductive member with the cutting member.

In another embodiment, the first electrically conductive member comprises a positive temperature coefficient material.

In another embodiment, the first jaw member further comprises an electrically insulative member positioned between the another jaw body member and the first electrically conductive member.

In another embodiment, the second jaw member further comprises an electrically insulative member positioned between the jaw body member and the second electrically conductive member.

In another embodiment, the electrosurgical instrument further comprises a sensor which is electrically coupled to the controller.

In another embodiment, the electrosurgical instrument further comprises a plurality of sensors electrically coupled to the controller.

In another embodiment, each of the first and second electrically conductive members is adapted to connect to an opposite potential of the electrosurgical energy source such that upon activation, the first and second electrically conductive members communicate bipolar energy through the tissue positioned between the first and second jaw members to effect the tissue seal.

In another embodiment, each of the first and second electrically conductive members is adapted to connect to an opposite potential of the electrosurgical energy source and the second jaw body member is connect to one potential of the electrosurgical energy source such that upon activation, the first and second electrically conductive members and the jaw body member communicate bipolar energy through the tissue positioned between the first and second jaw members to effect the tissue seal.

In another embodiment, the second electrically conductive member is selectively coupled with the first electrically conductive member and the second jaw body member based on a position of at least one of the first and second jaw members.

In one embodiment, an electrosurgical instrument is provided. The electrosurgical instrument comprises an end effector and a controller. The end effector is movable between a first position and a second position and comprises a first jaw member and a second jaw member. The first jaw member comprises a first plurality of electrically conductive members. Each of the first plurality of electrically conductive members are electrically isolated from one another. The second jaw member comprises a second plurality of electrically conductive members. Each of the second plurality of electrically conductive members are electrically isolated from one another. The first jaw member is movable relative to the second jaw member from an open position to a closed position to grasp a tissue positioned between the first and second jaw members. At least one of the first and second jaw members is adapted to connect to an electrosurgical energy source such that electrosurgical energy can be selectively communicated through the tissue positioned between the first and second jaw members to effect a tissue seal. The controller is configured to selectively electrically couple two electrically conductive members of the first plurality of electrically conductive members with one electrically conductive member of the second plurality of electrically conductive members.

In another embodiment, each of the first plurality of electrically conductive members are individually addressable, and each of the second plurality of electrically conductive members are individually addressable.

In another embodiment, at least one of the first plurality of electrically conductive members is offset from a corresponding electrically conductive member of the second plurality of electrically conductive members.

In another embodiment, the first plurality of electrically conductive members comprises a first electrically conductive member, a second electrically conductive member, and a third electrically conductive member. Each of the first, second and third electrically conductive members extend longitudinally and are oriented parallel relative to each other.

In another embodiment, the first plurality of electrically conductive members further comprises a fourth electrically conductive member. The fourth electrically conductive member is oriented transverse to the first, second and third electrically conductive members.

In another embodiment, the second plurality of electrically conductive members comprises a first electrically conductive member, a second electrically conductive member, and a third electrically conductive member. Each of the first, second and third electrically conductive members of the second plurality of electrically conductive members extend longitudinally and are oriented parallel relative to each other.

In another embodiment, the second plurality of electrically conductive members further comprises a fourth electrically conductive member. The fourth electrically conductive member of the second plurality of electrically conductive members is oriented transverse to the first, second and third electrically conductive members of the second plurality of electrically conductive members.

In another embodiment, the controller is further configured to selectively electrically couple one electrically conductive member of the first jaw member with two electrically conductive members of the second jaw member.

In another embodiment, the controller is further configured to concurrently selectively electrically couple a first one of the first plurality of electrically conductive members with a corresponding first one of the second plurality of electrically conductive members, and a second one of the first plurality of electrically conductive members with a corresponding second one of the second plurality of electrically conductive members.

In another embodiment, the controller is further configured to selectively couple one electrically conductive member of the first plurality of electrically conductive members with a corresponding electrically conductive member of the second plurality of electrically conductive members.

In another embodiment, the controller is further configured to cycle the electrosurgical instrument through a plurality of operating modes, wherein each respective operating mode is associated with a different combination of electrically coupled electrically conductive members of the first and second sets of electrically conductive members.

The foregoing summary is illustrative only and is not intended to be in any way limiting. In addition to the illustrative aspects, embodiments, and features described above, further aspects, embodiments, and features will become apparent by reference to the drawings and the following detailed description.

### FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 illustrates a simplified representation of an electrosurgical instrument according to various embodiments.

FIG. 2 is a perspective view of the electrosurgical instrument of FIG. 1 according to various embodiments.

FIG. 3 is a perspective view of the electrosurgical instrument of FIG. 1 according to various embodiments.

FIGS. 4 and 5 are partial perspective views of the electrosurgical instrument of FIG. 1 according to various embodiments.

FIG. 6 is an exploded view of various components of the electrosurgical instrument of FIG. 1 according to various embodiments.

FIG. 7 is a side view of the electrosurgical instrument of FIG. 1 according to various embodiments.

FIGS. 8-9 are perspective views of an end effector of the electrosurgical instrument of FIG. 1 according to various embodiments.

FIG. 10 is a side view of an end effector of the electrosurgical instrument of FIGS. 1-2 with the first and second jaw members in the open position, according to various embodiments.

FIG. 11 shows the closure bar and I-beam member of the electrosurgical instrument of FIGS. 1-2 at an initial stage of clamp closure and firing sequence where the I-beam member is located at the base of a ramp in the first jaw member, according to various embodiments.

FIG. 12 shows the closure bar and I-beam member further advanced distally than shown in FIG. 11, where the I-beam member is located at an intermediate position along the ramp in the first jaw member, according to various embodiments.

FIG. 13 shows the closure bar and I-beam member further advanced distally than shown in FIG. 12 where the I-beam member is located at the top of the ramp in the first jaw member, according to various embodiments.

FIG. 14 shows the closure bar and I-beam member further advanced distally than shown in FIG. 13, where the I-beam member is located past the ramp in the first jaw member, according to various embodiments.

FIGS. 15-16 illustrate various embodiments of an end effector of the electrosurgical instrument of FIG. 1.

FIGS. 17-18 illustrate side views of an end effector of the electrosurgical instrument of FIG. 1 according to various embodiments.

FIG. 19 is a simplified representation of electrical connections to a controller of the electrosurgical instrument of FIG. 1 according to various embodiments.

FIG. 20 illustrates other embodiments of the end effector of FIGS. 17-18.

FIG. 21 illustrates connections of the proximal end of electrical conductors of the electrosurgical instrument of FIG. 1 according to various embodiments.

FIG. 22 illustrates a simplified representation of various embodiments of a handle assembly of the electrosurgical instrument of FIG. 1 which can be utilized with the end effector of FIGS. 17-18 and 20.

FIG. 23 is a front view of the end effector of the electrosurgical instrument of FIG. 1 according to yet other embodiments.

FIG. 24 is a perspective view of the bottom of a first jaw member of the end effector of FIG. 23 according to various embodiments.

FIG. 25 is a perspective view of the top of a second jaw member of the end effector of FIG. 23 according to various embodiments.

### DESCRIPTION

In the following detailed description, reference is made to the accompanying drawings, which form a part hereof. In the drawings, similar symbols and reference characters typically identify similar components throughout the several views, unless context dictates otherwise. The illustrative embodiments described in the detailed description, drawings, and claims are not meant to be limiting. Other embodiments may be utilized, and other changes may be made, without departing from the scope of the subject matter presented here.

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Before explaining the various embodiments of the electrosurgical devices having controllable current paths in detail, it should be noted that the various embodiments disclosed herein are not limited in their application or use to the details of construction and arrangement of parts illustrated in the accompanying drawings and description. Rather, the disclosed embodiments may be positioned or incorporated in other embodiments, variations and modifications thereof, and may be practiced or carried out in various ways. Accordingly, embodiments of the surgical devices disclosed herein are illustrative in nature and are not meant to limit the scope or application thereof. Furthermore, unless otherwise indicated, the terms and expressions employed herein have been chosen for the purpose of describing the embodiments for the convenience of the reader and are not to limit the scope thereof. In addition, it should be understood that any one or more of the disclosed embodiments, expressions of embodiments, and/or examples thereof, can be combined with any one or more of the other disclosed embodiments, expressions of embodiments, and/or examples thereof, without limitation.

Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

In various embodiments, the present disclosure provides an electrosurgical radio frequency (RF) bipolar sealing and cutting device having controllable current paths. Different electric circuits within the device can be selectively coupled to a power supply to deliver energy to a tissue (e.g., a vessel) positioned between a set of opposing jaws of the device.

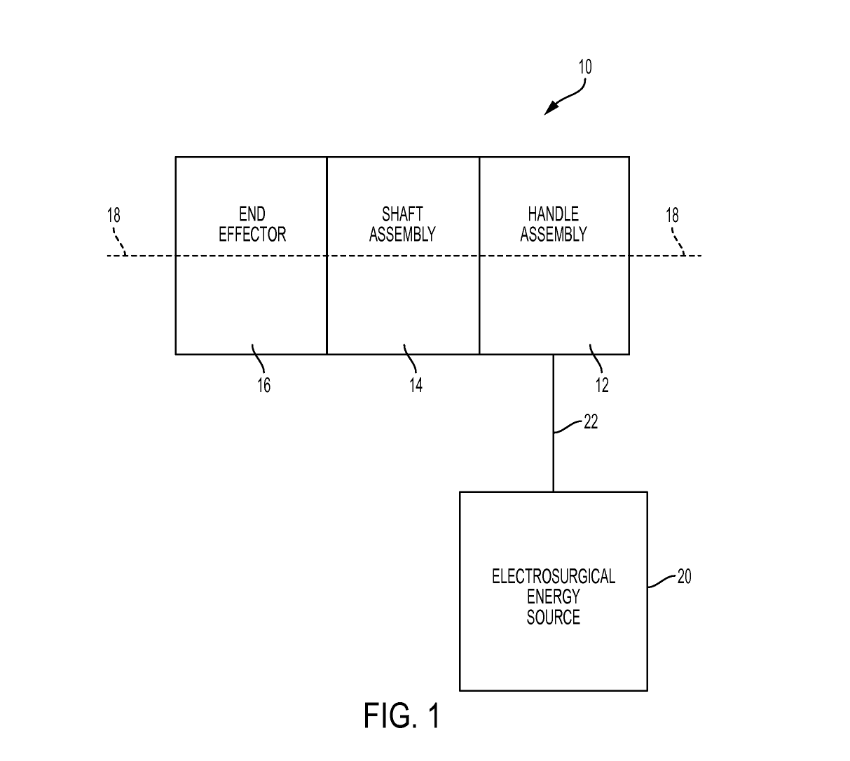

FIG. 1 illustrates a simplified representation of an electrosurgical instrument 10 according to various embodiments. The electrosurgical instrument 10 includes a handle assembly 12, a shaft assembly 14 and an end effector 16. As shown in FIG. 1, the electrosurgical instrument 10 defines a longitudinal axis 18 and may be coupled to an electrosurgical energy source 20 via an electrically conductive cable 22. The electrosurgical energy source 20 may be any type of electrosurgical energy source supply suitable for providing electrosurgical energy for therapeutic tissue treatment, tissue cauterization/sealing, as well as sub-therapeutic treatment and measurement. For example, according to various embodiments, the electrosurgical energy source 20 is a voltage supply which can provide electric current to the electrosurgical instrument 10, wherein the magnitude, duration, wave form, and/or frequency, for example, of the electric current can be sufficiently controlled or modulated to provide a desired amount of electrosurgical energy to the end effector 16 of the electrosurgical instrument 10.

The handle assembly 12 includes a proximal end and a distal end, the shaft assembly 14 includes a proximal end and a distal end, and the end effector 16 includes a proximal end and a distal end. The proximal end of the shaft assembly 14 is coupled to the distal end of the handle assembly 12, and the distal end of the shaft assembly 14 is coupled to the proximal end of the end effector 16. The shaft assembly 14 is rotatably connected to the handle assembly 12. Thus, the end effector 16 is also rotatable relative to the handle assembly 12. As described in more detail hereinbelow, the end effector 16 is movable between a first position (e.g., an “open” position) and a second position (e.g., a “closed” position). As used herein, the closed position can be any position other than the open position. For example, the closed position can be a fully closed position or a position between the open position and the fully closed position. In the open position, the end effector 16 is able to receive a tissue such as, for example, a vessel. As the end effector 16 is moved toward the fully closed position, the end effector 16 is able to apply a compressive force to the received tissue and deliver electrosurgical energy to the tissue. The electrosurgical energy delivered to the tissue may be, for example, RF energy, sub-therapeutic RF energy, ultrasonic energy and/or other suitable forms of energy.

As described in more detail hereinbelow, the electrosurgical instrument 10 can include various components and assemblies/systems which are operable to, for example, move the end effector 16 between the open and fully closed positions (a closure system), deliver electrosurgical energy to the end effector 16 (an energy delivery system), advance and retract a cutting member (a cutting member firing system), lockout a cutting member to prevent the cutting member from advancing and retracting (a cutting member lockout system), and lockout delivering electrosurgical energy to the end effector 16 (an energy lockout system).

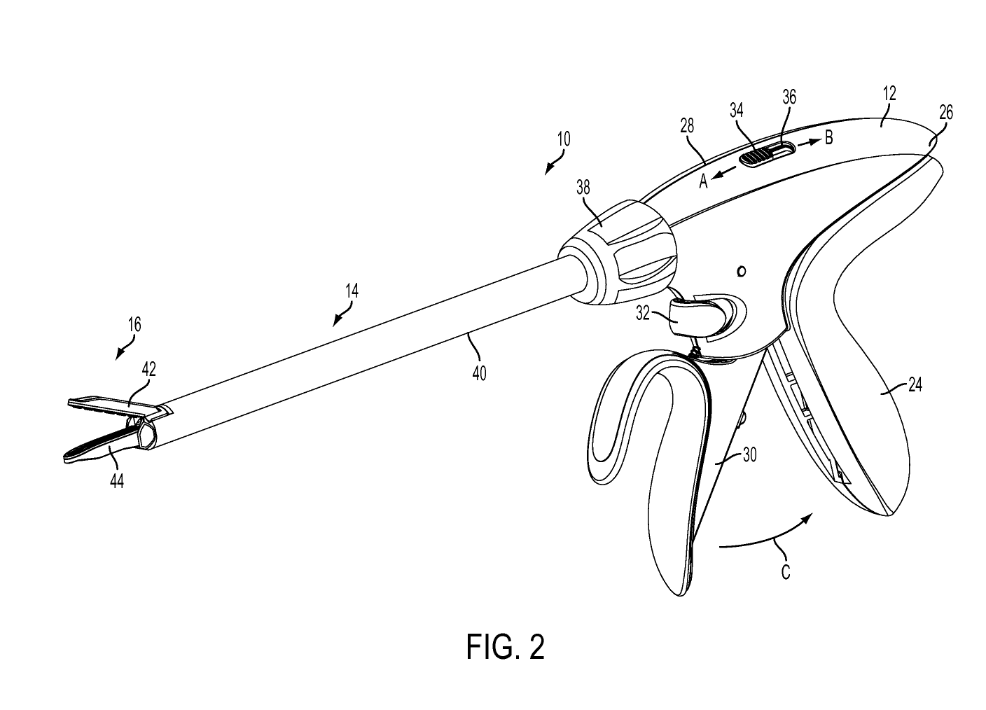

FIG. 2 is a perspective view of the electrosurgical instrument 10 according to various embodiments. The handle assembly 12 includes a pistol grip 24, a handle housing shroud 26 (e.g., the housing shroud shown on the “right” side of the handle assembly 12), a handle housing shroud 28 (e.g., the housing shroud shown on the “left” side of the handle assembly 12), a trigger 30 and an energy button 32. The trigger 30 is actuatable in the direction C towards the pistol grip 24 and assists in the control of the movement of the end effector 16 toward the closed position to enable the clamping of a tissue (e.g., a vessel). The energy button 32 operates to control the delivery of electrosurgical energy to the end effector 16, and thus may be considered as a component of the energy delivery system. The electrosurgical energy delivered to the end effector 16 may be, for example, RF energy, sub-therapeutic RF energy, ultrasonic energy and/or other suitable forms of energy.

As shown in FIG. 2, according to various embodiments, the handle assembly 12 may also include a lockout button 34 which is positioned within a slot 36 defined by the handle assembly 12. The lockout button 34 is movable between a first position A and a second position B, and is part of an optional cutting member lockout system which operates to lockout a cutting member to prevent the cutting member from advancing toward the distal end of the end effector 16.

As used herein, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, even though many un-biased buttons (due to their physical nature) require a spring to return to their un-pushed state. Terms for the “pushing” of the button, may include press, depress, mash, and punch.

The shaft assembly 14 includes a rotatable shaft knob 38 and an outer sheath 40. The rotatable shaft knob 38 is positioned at the proximal end of the shaft assembly 14 and is configured to rotate the shaft assembly 14 relative to the handle assembly 12. According to various embodiments, the distal end of the outer sheath 40 includes one or more contact electrodes (not shown) which are operatively coupled to the energy button 32.

The end effector 16 includes a first jaw member 42 (e.g., the jaw member shown as the “top” portion of the end effector 16) and a second jaw member 44 (e.g., the jaw member shown as the “bottom” portion of the end effector 16). Each of the first and second jaw members 42, 44 include a proximal end and a distal end. As described in more detail hereinbelow, at least one of the first and second jaw members 42, 44 are moveable relative to one another. In some embodiments, the first and second jaw members 42, 44 are each movable relative to the other. In other embodiments, first jaw member 42 is in a fixed position and the second jaw member 44 is movable relative to the first jaw member 42. In yet other embodiments, the second jaw member 44 is in a fixed position and the first jaw member 42 is movable relative to the second jaw member 44 as shown in FIG. 2. In various embodiments, the first jaw member 42 is movable relative to the second jaw member 44 from an open position to a closed position to grasp a tissue (not shown) therebetween, and at least one of the first and second jaw members 42, 44 is adapted to connect to the electrosurgical energy source 20 such that electrosurgical energy can be selectively communicated through the tissue positioned between the first and second jaw members 42, 44 to effect a tissue seal. According to various embodiments, each of the first and second jaw members 42, 44 is adapted to connect to an opposite potential of the electrosurgical energy source 20 such that upon activation, the jaw members 42, 44 conduct bipolar energy through the tissue positioned between the jaw members 42, 44 to effect the tissue seal.

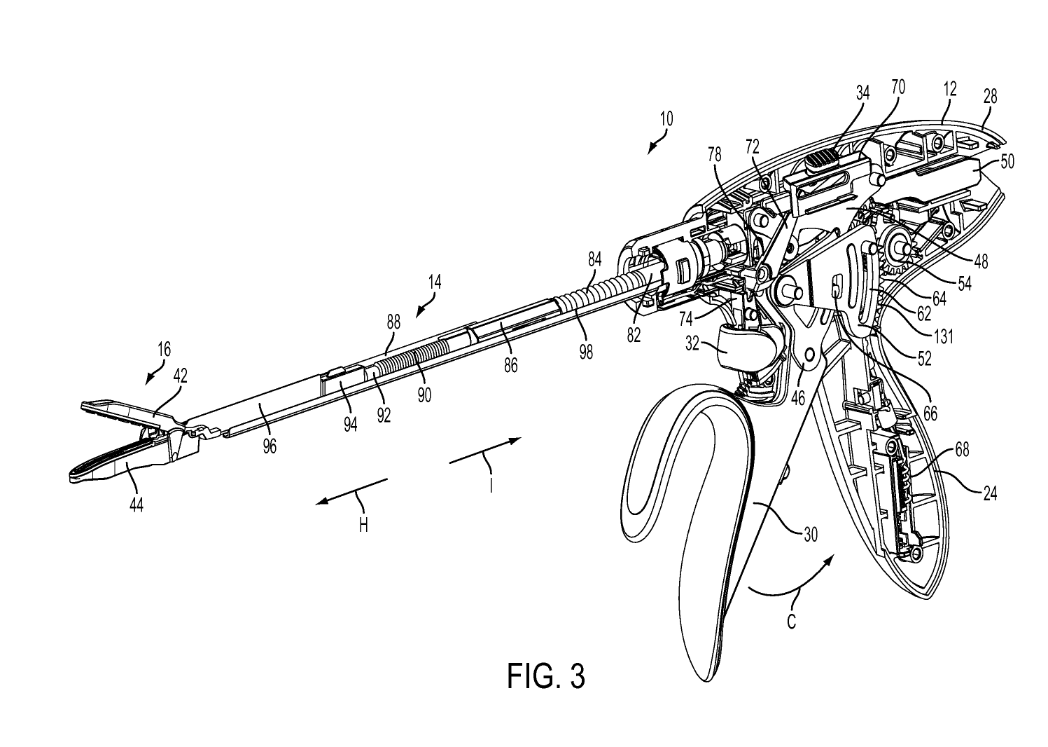

Regardless of the arrangement of the first and second jaw members 42, 44, the end effector 16 is movable between a first position and a second position. In the first position, which may be considered the open position, the first and second jaw members 42, 44 are spaced apart a maximum distance at their distal ends. In the first position, the end effector 16 is able to receive a tissue (not shown) such as, for example, a vessel, between the first and second jaw members 42, 44. In the second position, which may be considered the fully closed position, the first and second jaw members 42, 44 are spaced apart a minimum distance at their distal ends. In the second position, a tissue positioned between the first and second jaw members 42, 44 would be in a compressed state. Although not shown in FIG. 2 for purposes of simplicity, it will be appreciated that the end effector 16 also includes at least one electrically conductive member (e.g., an electrode configured to deliver electrosurgical energy to the tissue positioned between the first and second jaw members 42, 44) to deliver electrosurgical energy to a tissue positioned between the first and second jaw members 42, 44. The electrosurgical energy delivered by the electrically conductive member to the tissue may be, for example, RF energy, sub-therapeutic RF energy, ultrasonic energy and/or other suitable forms of energy. Additional components of the electrosurgical instrument 10 are described hereinbelow with respect to FIGS. 3-5.

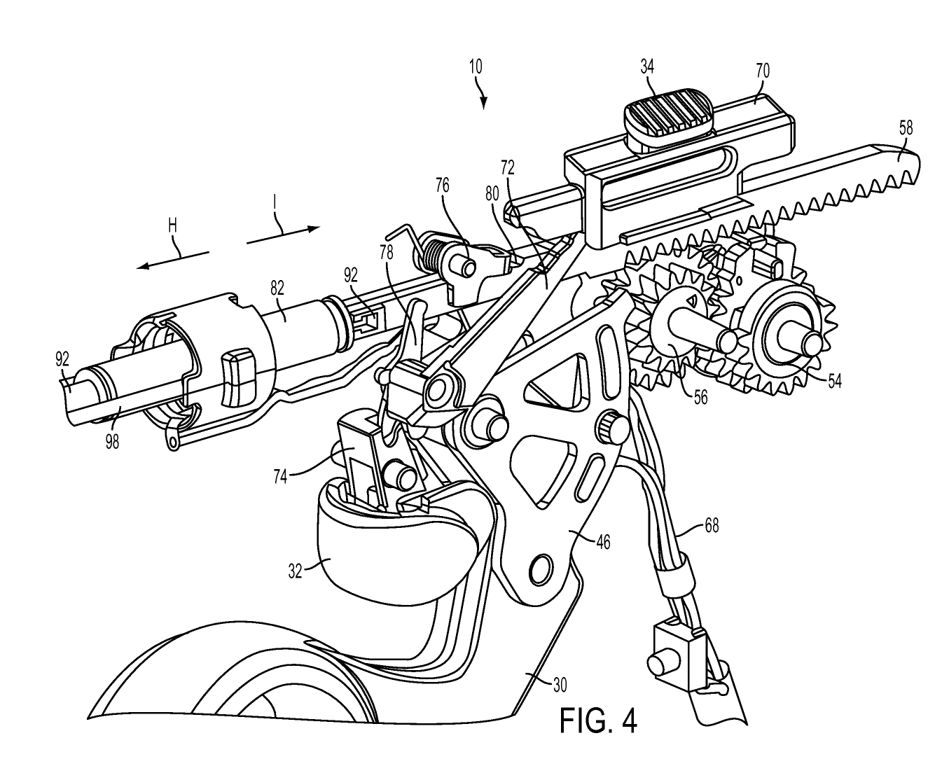

FIG. 3 is a perspective view of the electrosurgical instrument 10 according to various embodiments. To more clearly show additional components of the electrosurgical instrument 10, the housing shroud 26 of the handle assembly 12 and the outer sheath 40 of the shaft assembly 14 are not shown. As shown in FIG. 3, the handle assembly 12 also includes a trigger plate 46, a toggle clamp 48, a yoke 50, a firing plate 52 and a first pinion gear 54. Although hidden from view, the handle assembly 12 also includes a second pinion gear 56 (See FIG. 4), a rack 58 (See FIG. 4) and an energy switch (not shown) located behind or underneath the energy button 32.

The trigger plate 46 is operatively coupled to the trigger 30. Squeezing the trigger 30 in the direction C toward the pistol grip 24 rotates the trigger plate 46 which operates the toggle clamp 48 to advance the yoke 50 distally which assists in the movement of the end effector 16 toward the closed position. Thus, the trigger plate 46, the toggle clamp 48 and the yoke 50 may be considered as components of an end effector 16 closure system.

The firing plate 52, which may be embodied as a sector gear, defines a first slot 60, a second slot 62 and a plurality of teeth 64. The first slot 60 is configured to receive a pin 66 which is fixedly coupled to the trigger plate 46. Thus, the trigger plate 46 is operably coupled to the firing plate 52 and rotation of the trigger plate 46 causes a slight rotation of the firing plate 52. As the firing plate 52 rotates, the teeth 64 of the firing plate 52 engage with and rotate the first pinion gear 54. The rotation of the first pinion gear 54 causes the rotation of the second pinion gear 56 (See FIG. 4) which in turn causes the rack 58 (See FIG. 4) to advance toward the end effector 16. As described in more detail hereinbelow, the advancement of the rack 58 toward the end effector 16 can assist in the movement of a cutting member toward the distal end of the end effector 16. Thus, the firing plate 52, the first pinion gear 54, the second pinion gear 56 and the rack 58 may be considered as components of a cutting member firing system.

Although the electrically conductive cable 22 is not shown entering the housing assembly 12 for purposes of simplicity, one of the electrically conductive cables 68 which is positioned within the housing assembly 12 and is electrically coupled to the electrically conductive cable 22 is shown in FIG. 3. The housing assembly 12 may include any number of conductive cables 68 and each conductive cable 68 may be considered a component of the energy delivery system.

With regard to the optional cutting member lockout system, according to various embodiments the handle assembly 12 may also include a slide member 70, a lever arm 72, a lockout element 74, a lock arm 76 (See FIG. 4) and an unlock arm 78. The slide member 70 is connected to and follows any movement of the lockout button 34. When the lockout button 34 is located in position A, the lock arm 76, which may be seated in a notch 80 (See FIG. 4) of the rack 58, can be released by pressing or actuating the energy button 32. When the lock arm 76 is seated in the notch 80, the rack 58 is not able to advance distally. Pressing or actuating the energy button 32 rotates the lockout element 74, which in turn rotates the unlock arm 78 to release the lock arm 76. Once the lock arm 76 is released, the rack 58 is enabled to advance distally as the trigger 30 is moved in direction C further toward the pistol grip 24. When the lockout button 34 is moved to position B, the slide member 70 rotates the lever arm 72, which rotates the unlock arm 78 to releases the lock arm 76. While the lockout button 34 is in position B, the rack 58 can be advanced distally without the need to press the energy button 32 to rotate the lockout element 74. According to various embodiments, a detent may be provided to hold the button in either position A or B.

As shown in FIG. 3, the shaft assembly 14 also includes a closure actuator 82, a closing spring 84, a spring-to-bar interface member 86, a closure bar 88, an opening spring 90, a firing bar 92, a pusher block 94, a cutting member 96 and an electrically conductive member/cable 98. The closure actuator 82 is coupled to the distal end of the yoke 50 and the advancement of the yoke 50 toward the end effector 16 causes the closure actuator 82 to advance distally toward the end effector 16. The distal portion of the closure actuator 82 is sized to be received within the closing spring 84 and the proximal portion of the closure actuator 82 is sized to compress the closing spring 84. The closure actuator 82 operates on the closing spring 84 which is coupled to the spring-to-bar interface member 86 which is coupled to the closure bar 88. As described in more detail hereinbelow, the closure bar 88 is coupled to the end effector 16. Movement of the closure bar 88 in the direction toward the end effector 16 causes the end effector 16 to move toward the closed position. Thus, the closure actuator 82, the closing spring 84, the spring-to-bar interface member 86 and the closure bar 88 may be considered as components of the end effector 16 closure system. The opening spring 90 operates to bias the end effector 16 toward the open position and the trigger 30 away from the pistol grip 24.

The proximal end of the firing bar 92 is slidably received within the closure actuator 82 and is coupled to the distal end of the rack 58. The rack 58 is received within the yoke 50. The distal end of the firing bar 92 is coupled to the proximal end of the pusher block 94. The firing bar 92 is surrounded by the closing spring 84, the spring-to-bar interface member 86 and the opening spring 90. The distal end of the pusher block 94 is coupled to the cutting member 96. The cutting member 96 may be any type of cutting member suitable for cutting the tissue positioned between the first and second jaw members 42, 44. For example, according to various embodiments, the cutting member 96 may include a plurality of flexible bands which collectively form an I-beam shaped member having a cutting element at its distal end. According to various embodiments, the cutting member 96 includes an electrically conductive member (e.g., an electrode) for delivering electrosurgical energy such as, for example, RF energy, sub-therapeutic RF energy, ultrasonic energy and/or other suitable forms of energy to the tissue. As the rack 58 advances distally toward the end effector 16, the firing bar 92 and the pusher block 94 each advance distally toward the end effector 16, and the cutting member 96 advances distally through a slot or channel (not shown) formed in the first and second jaw members 40, 42. Thus, the firing bar 92, the pusher block 94 and the cutting member 96 may be considered as components of the cutting member firing system.

The electrically conductive cable/member 98 is electrically connected to a corresponding electrically conductive cable 68 in the housing assembly 12, and is utilized to delivery electrosurgical energy to the end effector 16. The shaft assembly 14 may include any number of electrically conductive cables/members 98, and each electrically conductive cable/member 98 may be considered as a component of the energy delivery system.

For the embodiments shown in FIG. 3, when the lockout button 34 is in the position A, the cutting member lockout system is enabled. When the cutting member lockout system is enabled, a user is forced to first move the end effector 16 towards to a closed/clamped position by squeezing the trigger 30 toward the pistol grip 24, then enable energy to be delivered to the end effector 16 by pressing the energy button 32, then enable the tissue positioned between the first and second jaw members 42, 44 to be cut by squeezing the trigger 30 further toward the pistol grip 24. According to various embodiments, in about the first thirteen degrees of stroke of the trigger 30 toward the pistol grip 24 (the first stroke), the end effector 16 is moved to a closed position. In about the last twenty-nine degrees of stroke of the trigger 30 toward the pistol grip 24 (the second stroke), the cutting member 96 is advanced to the distal end of the end effector 16. The cutting member 96 is locked out between the first stroke and the second stroke. If the energy button 32 is not pressed between the first and second strokes, the rack 58 and the firing bar 92 will not move distally toward the end effector 16 no matter how hard the trigger 30 is squeezed toward the pistol grip 24.

When the cutting member lockout system is enabled, the energy button 32 has to be depressed before the cutting member 96 can be released or the trigger 30 can move the rack 58 distally in the direction H. Thus, the energy button 32 functions as part of the cutting member lockout system as well as part of the energy delivery system. When the lockout button 34 is in the position B, the cutting member lockout system is disabled, and will remain disabled until the lockout button 34 is moved back to the position A. When the cutting member lockout system is disabled, the energy button 32 may appear to be depressed to provide a visual indication to a user that the cutting member lockout system has been disabled but without energizing the conductive members 96 in the end effector 16. When the cutting member lockout system is disabled, the cutting member 96 may be fired at will without the need to apply electrosurgical energy to one or more electrically conductive members (e.g., electrodes) in the end effector 16.

Although not shown in FIG. 3, in certain embodiments, the electrosurgical instrument 10 may include an automatic energy lockout system, and the energy lockout system can be associated with the end effector 16 closure system of the electrosurgical instrument 10. For such embodiments, the energy lockout system can be configured to permit energy delivery to the end effector 16 when the energy button 32 is actuated if the first and second jaw members 42, 44 are in the open position. In certain instances, the energy lockout system may be configured to deny electrosurgical energy delivery to the end effector 16 when the energy button 32 is actuated if the first and second jaw members 42, 44 are in a closed position. In certain instances, the energy lockout system operates to automatically transition from permitting the electrosurgical energy delivery to denying the electrosurgical energy delivery when the first and second jaw members 42, 44 are transitioned from the closed position to the open position, for example. In certain instances, the energy lockout system operates to automatically transition from denying the electrosurgical energy delivery to permitting the electrosurgical energy delivery when the first and second jaw members 42, 44 are transitioned from the open position to the closed position, for example.

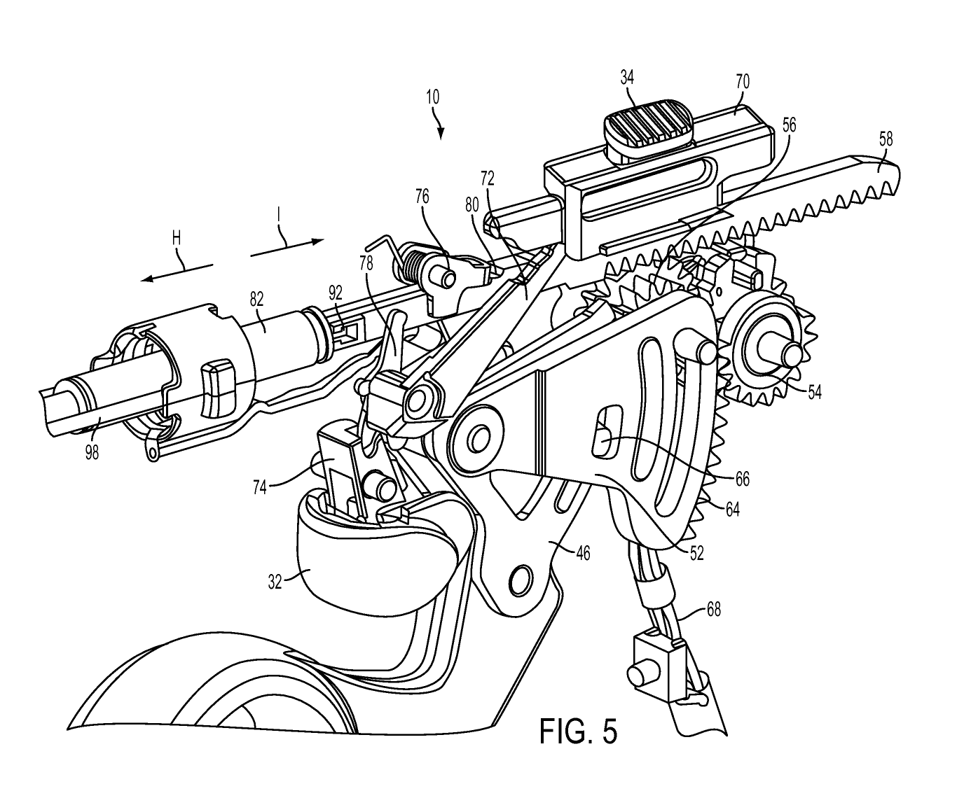

FIGS. 4 and 5 are partial perspective views of the electrosurgical instrument 10 according to various embodiments. To more clearly show the various components of the electrosurgical instrument 10, the handle housing shrouds 26, 28, the toggle clamp 48, the yoke 50 and the firing plate 52 are not shown in FIG. 4, but the firing plate 52 is shown in FIG. 5. In FIG. 4, the first pinion gear 54 is shown as being engaged with the second pinion gear 56, and the second pinion gear 56 is shown as being engaged with the rack 58. Additionally, the lock arm 76 is shown as being positioned in the notch 80 of the rack 58, and the proximal end of the firing bar 92 is shown as being coupled to the distal end of the rack 58. When the unlock arm 78 is in the indicated position, as the toggle clamp 48 and the yoke 50 move in the distal direction, the unlock arm 78 acts on the lock arm 76 to disengage the lock arm 76 from the notch 80 in the rack 58 to defeat the cutting member lockout system. Therefore, the rack 58 is able to advance distally when the firing plate 52 is rotated by the trigger 30. In FIG. 5, the relative positions of the firing plate 52, the first and second pinion gears 54, 56 and the rack 58 are shown prior to firing the cutting member.

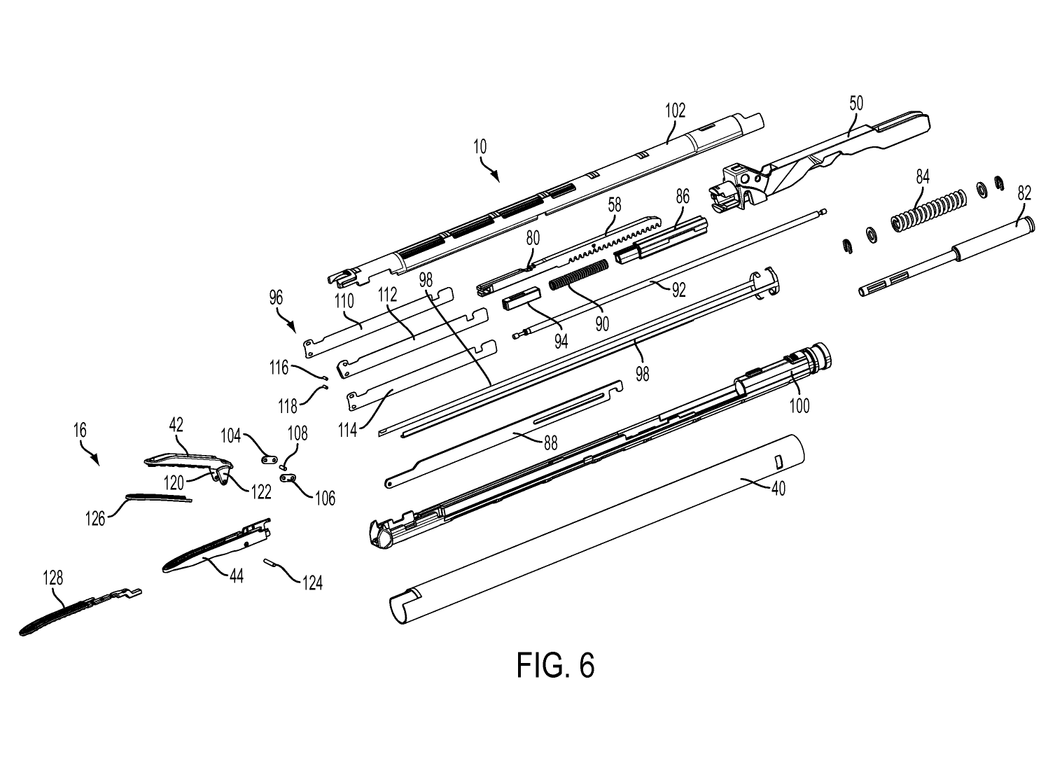

FIG. 6 is an exploded view of various components of the electrosurgical instrument 10 according to various embodiments. As shown in FIG. 6, the shaft assembly 14 further includes an electrically insulative nonconductive tube 100, a clamp tube 102, linkage members 104 and 106, pin 108, flexible bands 110, 112 and 114, and pins 116, 118. The electrically insulative nonconductive tube 100 is slidably received within the outer sheath 40 and contains or houses almost all of the various functional components of the shaft assembly 14. The electrically conductive cables/members 98 are located external to the electrically insulative nonconductive tube 100. The clamp tube 102 is connected to the electrically insulative nonconductive tube 100. The distal end of the closure bar 88 is coupled to the end effector 16 by the linkages 104, 106 and the pin 108. Although only one pin 108 is shown in FIG. 6, it will be appreciated that the electrosurgical instrument 10 may include two of pin 108 (e.g., 108a, 108b) and the distal end of the closure bar 88 can be coupled to the end effector 16 by the linkages 104, 106 and the pins 108a, 108b. The proximal ends of the flexible bands 110, 112, 114 are coupled to the pusher block 94 and are connected together at their distal ends by the pins 116, 118. The flexible bands 110, 112, 114 can collectively form various embodiments of the cutting member 96, where the cutting member 96 is an I-beam shaped cutting member having a cutting element as the distal ends of the flexible bands 110, 112, 114. Although three flexible bands 110, 112, 114 are shown in FIG. 6, it will be appreciated that the surgical instrument 10 may include any number of such flexible bands.

As also shown in FIG. 6, the end effector 16 further includes rotatable support members 120, 122, a pin 124 and electrically conductive members 126, 128 (e.g., electrodes). According to various embodiments, the first and second jaw members 42, 44 are pivotably coupled to one another via the rotatable support members 120, 122 and the pin 124. The electrically conductive members 126, 128 are electrically coupled to corresponding electrically conductive cables/members 98 in the shaft assembly 14, and are utilized to deliver electrosurgical energy to a tissue (e.g., a vessel) positioned between the first and second jaw members 42, 44. Although only one electrically conductive member 126 associated with the first jaw member 42 and one electrically conductive member 128 associated with the second jaw member 44 are shown in FIG. 6, it will be appreciated that the first and/or second jaw members 42, 44 may include any number of electrically conductive members, and each electrically conductive member may be considered as a component of the energy delivery system. As described hereinabove, the electrosurgical energy delivered to the tissue by the electrically conductive member 126 and/or the electrically conductive member 128 may be, for example, RF energy, sub-therapeutic RF energy, ultrasonic energy and/or other suitable forms of energy.

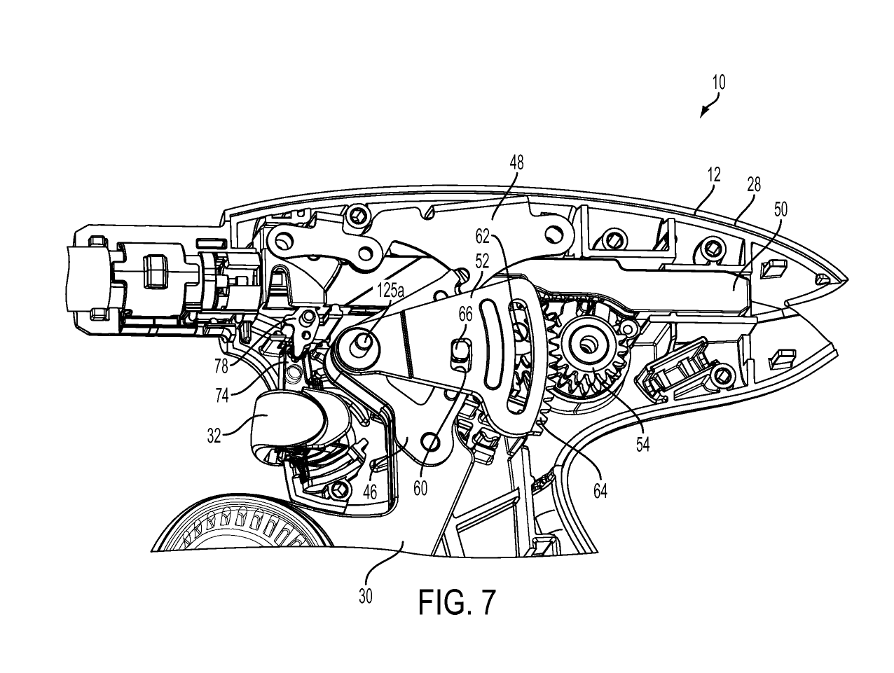

FIG. 7 is a side view of the electrosurgical instrument 10 according to various embodiments. To more clearly show the various components of the electrosurgical instrument 10, the first housing shroud 26, the lockout button 34, the conductive cable 68 and the slide member 70 are not shown. Of course, according to various embodiments, the electrosurgical instrument 10 may not include the lockout button 34, the slide member 70 and/or the cutting member lockout system. For embodiments which do not include the cutting member lockout system, except for the cutting member lockout system, the electrosurgical instrument otherwise operates the same as the electrosurgical instrument 10.

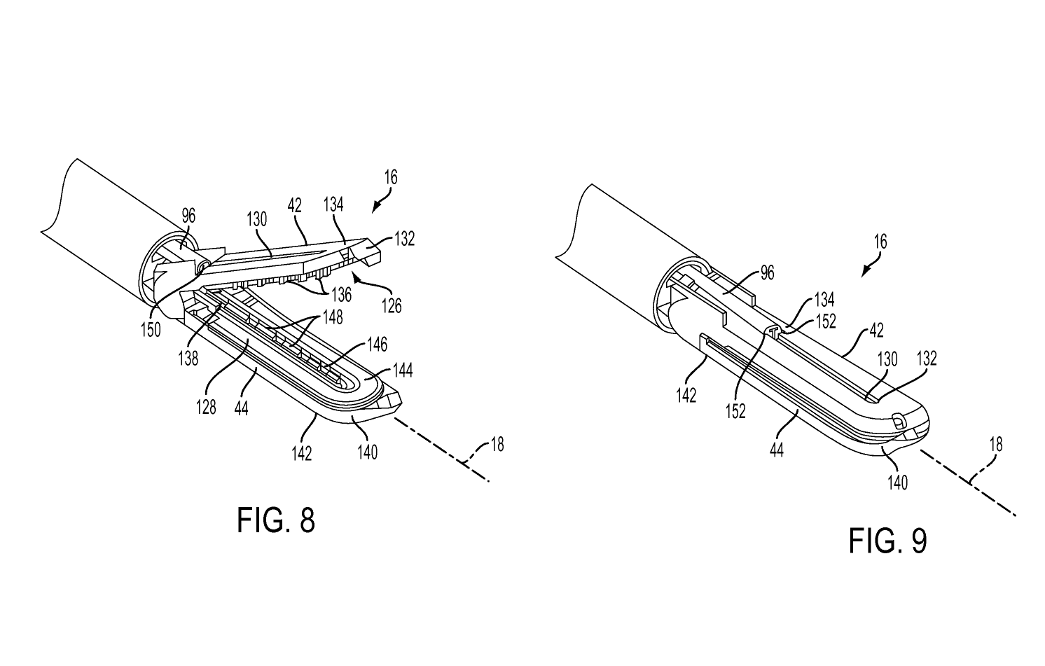

FIGS. 8-9 are perspective views of the end effector 16 according to various embodiments. The end effector 16 may be used with the electrosurgical instrument 10 or with any other any suitable electrosurgical instrument. Although the end effector 16 shown in FIGS. 8-9 is axially aligned with the longitudinal axis 18, according to other embodiments the end effector 16 may be curved and the majority of the end effector 16 may not be axially aligned with the longitudinal axis 18. FIG. 8 shows the end effector 16 in the open position, and also shows an elongate slot or channel 130 defined by the first jaw member 42. The elongate slot 130 may be axially aligned with the longitudinal axis 18. The first jaw member 42 includes a jaw body member 132 which includes a surface 134 (shown as a “top” surface of the jaw body member 132). The first jaw member 42 also includes the electrically conductive member 126 which includes an energy delivery surface 136 (shown as a “bottom” surface of the electrically conductive member 126). The energy delivery surface 136 may be configured as a U-shaped surface that extends about the distal end of the first jaw member 42. FIG. 8 also shows an elongate slot or channel 138 defined by the second jaw member 44. The elongate slot 138 may be axially aligned with the longitudinal axis 18. The second jaw member 44 includes a jaw body member 140 which includes a surface 142 (shown as a “bottom” surface of the jaw body member 140). The second jaw member 44 also includes the electrically conductive member 128 which includes an energy delivery surface 144 (shown as a “top” surface of the conductive member 128). The energy delivery surface 144 may be configured as a U-shaped surface that extends about the distal end of the second jaw member 44.

According to various embodiments, the first jaw member 42 and/or the second jaw member 44 may include a surface adjacent and/or near the elongate slots. For example, see the surface 146 adjacent the elongate slot 138. The surface 146 can include a thermally and/or electrically conductive material such as, for example, a metal. In some embodiments, the surface 146 may be integral with or in direct or indirect contact with the jaw body member 140 to conduct heat and/or electricity away from a tissue. According to various embodiments, the surface 146 can define a plurality of teeth 148 which can be utilized to grip a tissue positioned between the first and second jaw members 42, 44. With regard to the cutting member 96, the cutting member 96 may include a distal blade 150 for cutting the tissue.

FIG. 9 shows the end effector 16 in the closed position. The cutting member 96 may be sized and configured to fit at least partially within the slots/channels 130, 138. The cutting member 96 may translate along the slots/channels 130, 138 between a first, retracted position which corresponds to the end effector 16 being in the open position (FIG. 8), and a second, advanced position which corresponds to the end effector 16 being in the closed position (FIG. 9). The cutting element 96 and/or the distal blade 150 may include any suitable material. For example, according to various embodiments, the cutting element 96 and/or the distal blade 150 may include 17-4 precipitation hardened stainless steel. At least a portion of the cutting member 96 may be 716 stainless steel. The distal portion of the cutting member 96 may comprise a flanged “I”-beam configured to slide within the slots/channels 130, 138 in the first and second jaw members 42, 44. In various embodiments, the distal portion of the cutting member 96 may comprise a “C”-shaped beam configured to slide within one of slots/channels 130, 138. As illustrated in FIG. 9, the cutting member 96 can reside in and/or on the slot/channel 130 of the first jaw member 42. The cutting member 96 may slide within the slot/channel 130, for example, to open and close the first jaw member 42 with respect to the second jaw member 44. The distal portion of the cutting member 96 also may define cam surfaces 152 for the surface 134 of the first jaw member 42, for example. Accordingly, as the cutting member 96 is advanced distally through the slot/channel 130, from, for example, a first position (FIG. 8) to a second position (FIG. 9), the first jaw member 42 may be urged closed (FIG. 9). The I-beam or the C-beam closure system described hereinabove may be utilized in connection with or in lieu of the end effector 16 closure system described hereinabove which includes the closure bar 88 and the linkage members 104, 106.

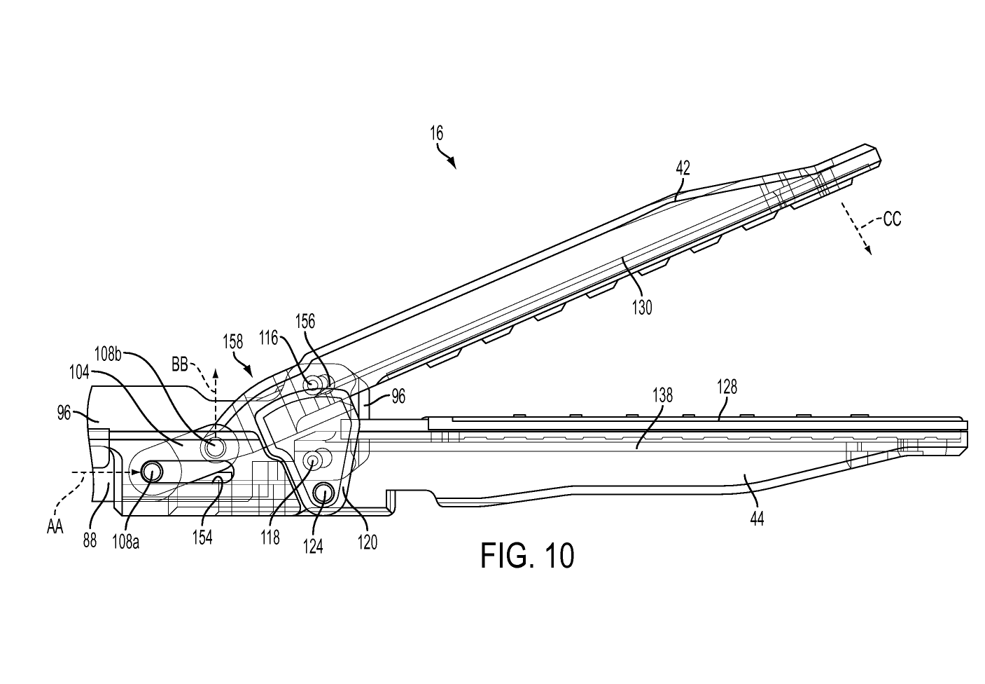

FIG. 10 is a side view of an end effector 16 of the electrosurgical instrument 10 shown in FIGS. 1 and 2 with the first and second jaw members 42, 44 in the open position, according to various embodiments. As shown in FIG. 10, the closure bar 88 defines a slot 154, the first jaw member 42 defines a ramp 156, and the flexible bands 110, 112, 114 and the pins 116, 118 collectively form an I-beam member 158. The closure bar 88 is operatively coupled to the proximal end of the first jaw member 42 via the closure linkages 104, 106 (not shown) and the pins 108a, 108b. The lower pin 108a is slidably movable within the slot 154. As the closure bar 88 moves distally in the direction indicated by arrow AA, the pin 108a slides in the slot 154 distally and forces the pin 108b to move upwardly in the direction indicated by arrow BB to force the first jaw member 42 to rotate toward a closed position as indicated by arrow CC. The first jaw member 42 pivots about a pivot point defined by the fastener pin 124. The second jaw member 44 includes the electrically conductive member 128, which is electrically coupled to the electrosurgical energy source 20. The I-beam member 158 forces the first and second jaw members 42, 44 to close when the cutting member 96 is fired by the rack 58 and the firing bar 92 as previously described. The I-beam member 158 advances distally on the slots/channels 130, 138 of the first and second jaw members 42, 44 to force the first and second jaw members 42, 44 shut and compress the tissue positioned therebetween. The ramp 156 is defined at the proximal end of the slot/channel 130 in the first jaw member 42. Accordingly, a predetermined force is required to advance the I-beam member 158 over the ramp 156 before the I-beam member 158 engages the slot/channel 130 to close the first and second jaw members 42, 44 as the I-beam member 158 is advanced distally by the flexible bands 110, 112, 114. In FIG. 10, the I-beam member 158 is located behind the ramp 154 as the linkage members 104, 106 (not shown) close the first and second jaw members 42, 44.

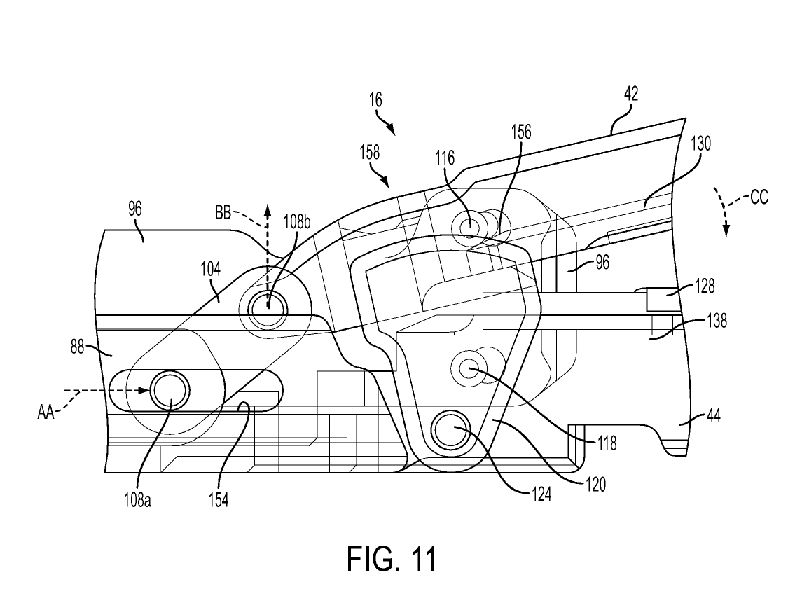

FIGS. 11-14 illustrate a sequence of firing the I-beam member 158 and closure spring 84 driven cam system to simultaneously close the first and second jaw members 42, 44. FIG. 11 shows the closure bar 88 and the I-beam member 158 at the initial stage of clamp closure and firing sequence where the I-beam member 158 is located proximal to (behind or at the base of) the ramp 156, according to various embodiments. The pins 116, 118 of the I-beam member 158 are located at the base of the ramp 156 prior to firing the cutting member 96. In FIG. 11, the I-beam member 158 is located behind the ramp 156 as linking member 120 closes the first jaw member 42 in the direction CC.

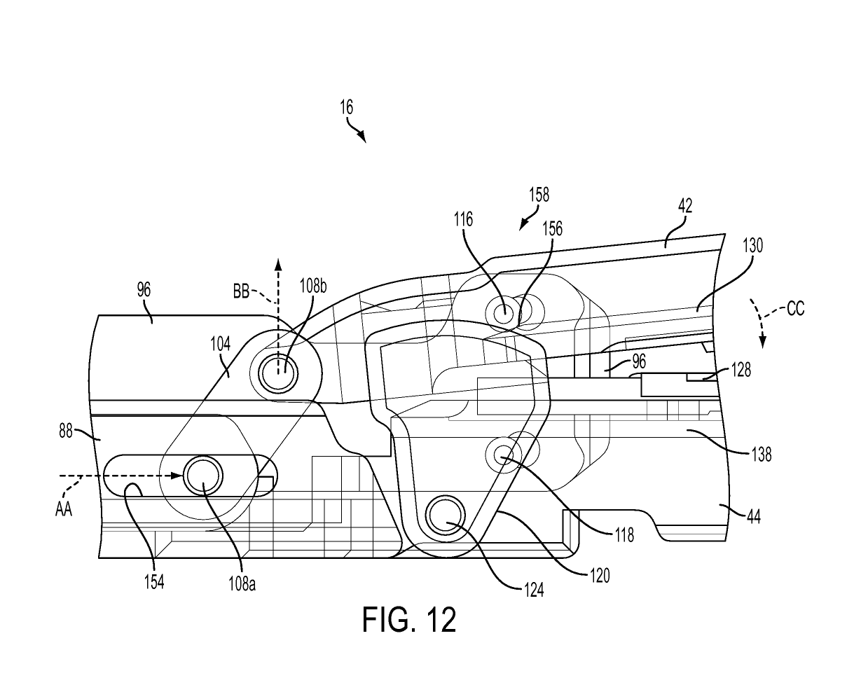

FIG. 12 shows the closure bar 88 and I-beam member 158 located at an intermediate position along the ramp 156 in the first jaw member 42 (the I-beam member 158 is further advanced distally in the direction AA than shown in FIG. 11), according to various embodiments. FIG. 12 shows the closure bar 88 pushing on the bottom pin 108a to move distally in direction AA within the slot 154. In response, the pivoting member 104 moves distally in direction AA and rotates counterclockwise pushing the pin 108b upwardly in the direction BB to apply a closing force to the first jaw member 42. The I-beam member 158 also advances partially up the ramp 156. The first jaw member 42 rotates slightly in direction CC toward a closed position.

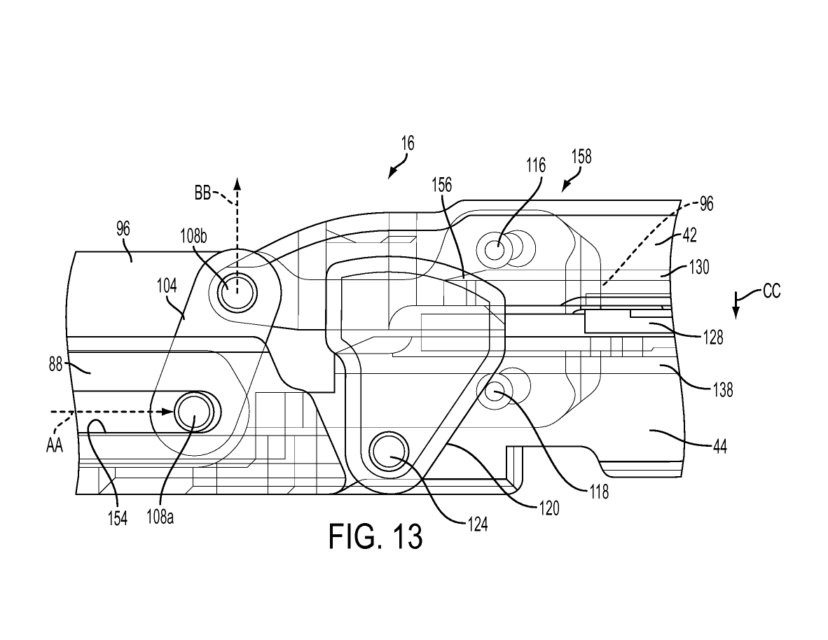

FIG. 13 shows the closure bar 88 and the I-beam member 158 further advanced distally in the direction AA than shown in FIG. 12, and shows the I-beam member 158 is located at the top of the ramp 204, according to various embodiments. In FIG. 13, the closure bar 88 is advanced further distally in direction AA in response to the closure actuator 82 acting on the closing spring 84 and continues pushing on the bottom pin 108a causing it to move further distally in the direction AA within the slot 154. In response, the pivoting member 104 moves distally in the direction AA and continues rotating counterclockwise pushing the pin 108b upwardly in the direction BB to apply a closing force to the first jaw member 42. The first jaw member 42 continues rotating further in the direction CC toward a closed position. At this stage, the I-beam member 158 is located at the top of the ramp 156.

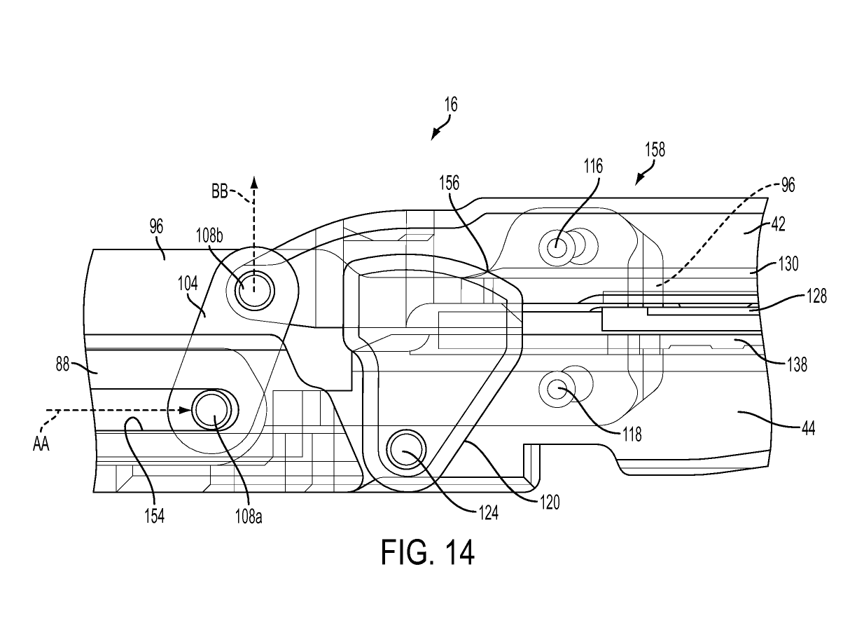

FIG. 14 shows the closure bar 88 and the I-beam member 158 further advanced distally than shown in FIG. 13, and shows the I-beam member 158 located past the ramp 156, according to various embodiments. FIG. 14 shows the closure bar 88 advanced still further distally in the direction AA and continues to push on the pin 108a causing it to move distally in the direction AA within the slot 154. In response, the pivoting member 104 moves distally in the direction AA and continues rotating counterclockwise pushing the pin 108b upwardly in the direction BB to apply a closing force to the first jaw member 42. The first jaw member 42 continues rotating further in the direction CC toward a closed position. In FIG. 14, the I-beam member 158 is located past the ramp 156 and the end effector 16 is fully closed in response to the trigger plate 46 acting on the toggle clamp 48, which acts on the yoke 50, and advances the closure actuator 82 and the closure bar 88 to push on the pivoting member 104. The pins 116, 118 of the I-beam member 158 are now located past the ramp 156 and are located in the slots/channels 130, 138 formed in the respective first and second jaw members 42, 44. The I-beam member 158 is now prepared to slide distally in the direction AA. In response to the trigger 30 being squeezed, the firing plate 52 rotates to advance the rack 58 distally, which acts on the firing bar 92 and pushes the I-beam member 158 and the cutting member 96 distally in the direction AA. This action forces the first and second jaw members 42, 44 fully shut to compress the tissue located therebetween.

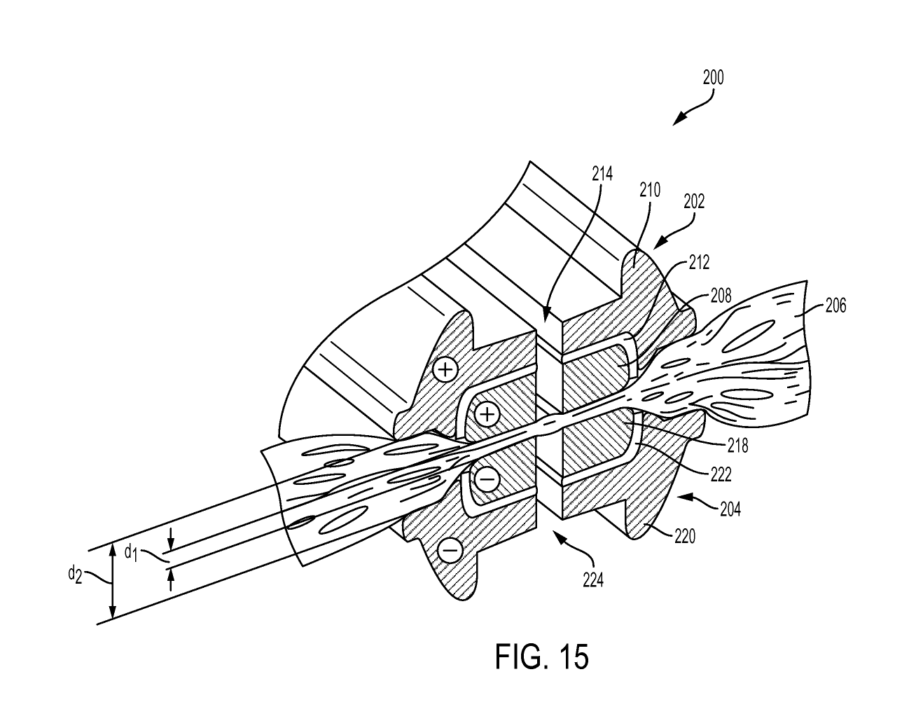

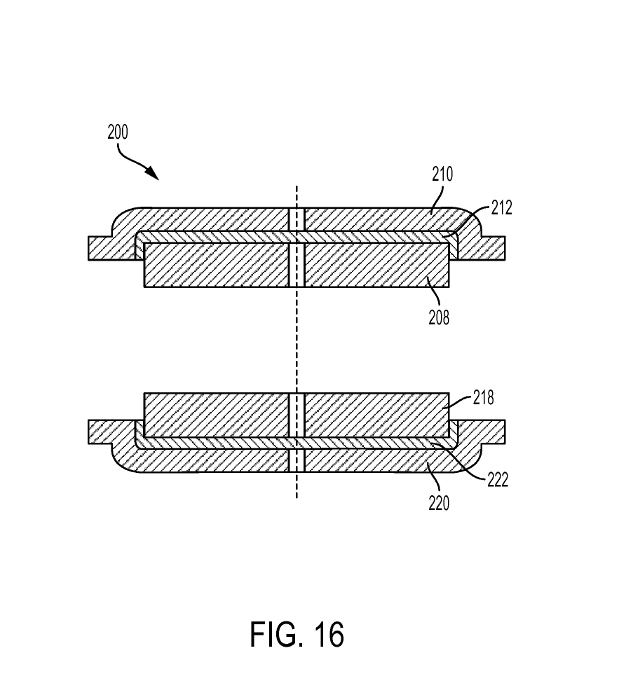

FIGS. 15-16 illustrate various embodiments of an end effector 200. The end effector 200 may be used in lieu of the end effector 16 with the electrosurgical instrument 10, and may also be used with any other any suitable electrosurgical instrument. Although the end effector 200 shown in FIGS. 15-16 is axially aligned with the longitudinal axis 18, according to other embodiments the end effector 200 may be curved and the majority of the end effector 200 may not be axially aligned with the longitudinal axis 18. Except for certain differences noted hereinbelow, the end effector 200 is similar to the end effector 16.

FIG. 15 illustrates a perspective view of an end effector 200 according to various embodiments. The end effector 200 includes a first jaw member 202 and a second jaw member 204. As explained in more detail hereinbelow, the first and second jaw members 202, 204 are configured to apply different compressive forces and deliver the same or different levels of electrosurgical energy to different portions of a tissue (e.g. a vessel) 206 positioned between the first and second jaw members 202, 204.

At least one of the first and second jaw members 202, 204 are movable relative to one another in a manner identical or similar to that of the first and second jaw members 42, 44 described hereinabove. In some embodiments, the first and second jaw members 202, 204 are each movable relative to the other. In other embodiments, first jaw member 202 is in a fixed position and the second jaw member 204 is movable relative to the first jaw member 202. In yet other embodiments, the second jaw member 204 is in a fixed position and the first jaw member 202 is movable relative to the second jaw member 204. Regardless of the arrangement of the first and second jaw members 202, 204, the end effector 200 is movable between a first position and a second position. In the first position, which may be considered an open position, the first and second jaw members 202, 204 are spaced apart a maximum distance at their distal ends. In the first position, the end effector 200 is able to receive a tissue 206 (e.g., a vessel) between the first and second jaw members 202, 204. In the second position, which may be considered a fully closed position, the first and second jaw members 202, 204 are spaced apart a minimum distance at their distal ends. As the end effector 200 is moved from the open position toward the fully closed position, the end effector 200 is able to apply compressive forces to the received tissue 206 and deliver electrosurgical energy to the tissue 206. The electrosurgical energy delivered to the tissue 206 may be, for example, RF energy, sub-therapeutic RF energy, ultrasonic energy and/or other suitable forms of energy. According to various embodiments, each of the first and second jaw members 202, 204 is adapted to connect to an opposite potential of the electrosurgical energy source 20 such that upon activation, the jaw members 202, 204 conduct bipolar energy through the tissue positioned between the jaw members 202, 204 to effect the tissue seal.

The first jaw member 202 includes a first electrically conductive member 208 (e.g., an electrode configured to deliver electrosurgical energy to the tissue positioned between the first and second jaw members 202, 204), a second electrically conductive member 210 (e.g., an electrode) and an electrically insulative member 212 positioned between the first and second electrically conductive members 208, 210. Additionally, according to various embodiments, the first jaw member 202 may define a slot or channel 214, and the channel 214 may be axially aligned with the longitudinal axis 18. The first electrically conductive member 208 is electrically isolated from the second electrically conductive member 210 by the electrically insulative member 212. Although not shown for purposes of simplicity in FIG. 15, it will be appreciated that the first and second electrically conductive members 208, 210 are electrically coupled to the electrosurgical energy source 20. The first and second electrically conductive members 208, 210 may include any suitable electrically conductive material. For example, according to various embodiments, the first electrically conductive member 208 may include an electrically conductive metal, an electrically conductive alloy, an electrically conductive polymer, a positive temperature coefficient material which has variable electrical conductivity, combinations thereof, etc. The material in the second electrically conductive member 210 may be the same as or different than the material in the first electrically conductive member 208. The electrically insulative member 212 may include any suitable type of electrically insulative material. For example, according to various embodiments, the electrically insulative member 212 includes a ceramic electrically insulative material, a polymer electrically insulative material, combinations thereof, etc.

According to various embodiments, the first jaw member 202 may also include a housing member 216 (not shown) which holds and/or supports the first and second electrically conductive members 208, 210 and the electrically insulative member 212. The housing member 216 may be formed from any suitable non-conductive material. For example, according to various embodiments, the housing member 216 includes a ceramic zirconia material.

The second jaw member 204 includes a third electrically conductive member 218 (e.g., an electrode), a fourth electrically conductive member 220 (e.g., an electrode) and an electrically insulative member 222 positioned between the third and fourth electrically conductive members 218, 220. Additionally, according to various embodiments, the second jaw member 204 may define a slot or channel 224, and the channel 224 may be axially aligned with the longitudinal axis 18. The third electrically conductive member 218 is electrically isolated from the fourth electrically conductive member 220 by the electrically insulative member 222. Although not shown for purposes of simplicity in FIG. 15, it will be appreciated that the third and fourth electrically conductive members 218, 220 are also electrically coupled to the electrosurgical energy source 20. The third and fourth electrically conductive members 218, 220 may include any suitable electrically conductive material. The electrically conductive material in the third electrically conductive member 218 may be the same as or different than the electrically conductive material in the fourth electrically conductive member 220, and the electrically conductive material in the third and/or fourth electrically conductive members 218, 220 may be the same as or different than the electrically conductive material in the first and/or second electrically conductive members 208, 210. The electrically insulative member 222 may include any suitable type of electrically insulative material, and the electrically insulative material in the electrically insulative member 222 may be the same as or different than the electrically insulative material in the electrically insulative member 212.

According to various embodiments, the second jaw member 204 may also include a housing member 226 (not shown) which holds and/or supports the third and fourth electrically conductive members 218, 220 and the electrically insulative member 222. The housing member 226 may be formed from any suitable non-conductive material, and the non-conductive material in the housing member 226 may be the same as or different than the non-conductive material in the housing member 216.

For embodiments of the end effector 200 which include the channels 214, 224, the channels 214, 224 cooperate to provide a path for an I-beam member or a C-beam member (neither of which are shown for purposes of simplicity) to advance from a position proximate the proximal end of the end effector 200 to a position proximate the distal end of the end effector 200. The I-beam member may be identical or similar to the I-beam member 158, the C-beam member may be identical or similar to the C-beam member described hereinabove, and either may be advanced distally in a manner the same as or different from that described hereinabove. In general, as the I-beam shaped member advances from a position proximate the proximal end of the end effector 200 to a position proximate the distal end of the end effector 200, the I-beam shaped member compresses (or further compresses) the portions of the tissue 206 positioned between the first and second jaw members 202, 204 and may also cut through the tissue 206. For embodiments which do not include the channels 214, 224 and the I-beam or C-beam member, the electrosurgical instrument 10 utilized with the end effector 200 may include other types of channels (e.g., a knife channel), cutting and/or closure systems.

As shown in FIG. 15 the geometry of the first and second jaw members 202, 204 is such that in a plane which is transverse to the longitudinal axis 18, a minimum distance (represented as d₁) between the first and third electrically conductive members 208, 218 is less than a minimum distance (represented as d₂) between the second and fourth electrically conductive members 210, 220. This is the case regardless of whether the end effector 200 is in the open position, the fully closed position or between the open and fully closed positions. Although the embodiments of the end effector 200 shown in FIG. 15 illustrate exemplary geometries of the first and second jaw members 202, 204, it will be appreciated that any number of different geometries may be utilized to realize the distance between the first and third electrically conductive members 208, 218 being less than the distance between the second and fourth electrically conductive members 210, 220 at a given point along the longitudinal axis 18. For example, FIG. 16 illustrates other embodiments of the end effector 200, where the geometries of the first and second jaw members 202, 204 differ from those shown in FIG. 15.

In operation, when the end effector 200 is placed into the open position, the tissue 206 can be positioned between the first and second jaw members 202, 204. As the end effector 200 is moved toward the closed position, the first and third electrically conductive members 208, 218 eventually make contact with and cooperate to apply a compressive force to the portion of the tissue 206 positioned between the first and third electrically conductive members 208, 218. At this point, the first electrically conductive member 208 can be selectively coupled to the electrosurgical energy source 20 to have a first level of electrosurgical energy applied to the first electrically conductive member 208. According to various embodiments, the first level of energy may range from 50 to 300 watts with a voltage limit ranging from 20 to 100 volts RMS and a current limit ranging from 1 to 4 amperes RMS. According to various embodiments, the first level of energy is 100 watts maximum with a voltage limit of 100 volts RMS and a current limit of 3 amperes RMS where the power, voltage and current limits are not exceeded. The first level of electrosurgical energy may be embodied in any suitable type of waveform applied to the first electrically conductive member 208. For example, according to various embodiments, the waveform is a sinusoid. Due to the difference in electric potential between the first and third electrically conductive members 208, 218, high frequency current is induced to flow (1) from the first electrically conductive member 208 to the portion of the tissue 206 positioned between the first and third electrically conductive members 208, 218, (2) through the portion of the tissue 206 positioned between the first and third electrically conductive members 208, 218 and (3) from the portion of the tissue 206 positioned between the first and third electrically conductive members 208, 218 to the third electrically conductive member 218. The high frequency current passing through the portion of the tissue 206 positioned between the first and third electrically conductive members 208, 218 operates to heat and seal the portion of the tissue 206 positioned between the first and third electrically conductive members 208, 218. Of course, since the induced high frequency current is alternating current, it will be appreciated that the induced high frequency current is also induced to flow in a path which is “reverse” to that described hereinabove.

As the end effector 200 is moved further toward the closed position, at some point, the third and fourth electrically conductive members 210, 220 make contact with and cooperate to apply a compressive force to the portions of the tissue 206 positioned between the third and fourth electrically conductive members 210, 220. At this point, the third electrically conductive member 210 can be selectively coupled to the electrosurgical energy source 20 to apply a second level of electrosurgical energy to the second electrically conductive member 210. According to various embodiments, the second level of energy may range from 20 to 100 watts with a voltage limit ranging from 20 to 100 volts RMS and a current limit ranging from 1 to 2 amperes RMS. According to various embodiments, the second level of energy is 100 watts maximum with a voltage limit of 100 volts RMS and a current limit of 3 amperes RMS where the power, voltage and current limits are not exceeded. Thus, it will be appreciated that the second level of electrosurgical energy applied to the second electrically conductive member 210 can be substantially the same as, less than or greater than the first level of electrosurgical energy applied to the first electrically conductive member 208. The second level of electrosurgical energy may be embodied in any suitable type of waveform applied to the second electrically conductive member 210, and the waveform may be the same as or different than the type of waveform applied to the first electrically conductive member 208. For example, according to various embodiments, the waveform is a sinusoid. Due to the difference in electric potential between the second and fourth electrically conductive members 210, 220, a high-frequency current is induced to flow (1) from the second electrically conductive member 210 to the portion of the tissue 206 positioned between the second and fourth electrically conductive members 210, 220, (2) through the portion of the tissue 206 positioned between the second and fourth electrically conductive members 210, 220 and (3) from the portion of the tissue 206 positioned between the second and fourth electrically conductive members 210, 220 to the fourth electrically conductive member 220. The high frequency current passing through the portion of the tissue 206 positioned between the second and fourth electrically conductive members 210, 220 operates to non-destructively bond the portions of the tissue 206 positioned between the second and fourth electrically conductive members 210, 220. Of course, since the induced high-frequency current is alternating current, it will be appreciated that the induced high-frequency current is also induced to flow in a path which is “reverse” to that described hereinabove. Because the minimum distance between the second and fourth electrically conductive members 210, 220 is greater than the minimum distance between the first and third electrically conductive members 208, 218, it will be appreciated that for a given level of electrosurgical energy applied to tissue positioned between the first and second jaw members 202, 204, the effect on the portion of the tissue positioned between the second and fourth electrically conductive members 210, 220 can be different from the effect on the portion of the tissue positioned between the first and third electrically conductive members 208, 218.

According to various embodiments, the level of the electrosurgical energy applied to the first electrically conductive member 208 and the level of electrosurgical energy applied to the second electrically conductive member 210 can be selected external to the electrosurgical instrument 10. For example, when it is desired to apply the first level of electrosurgical energy to the first electrically conductive member 208, the electrosurgical energy source 20 can be set to a first mode of operation. Similarly, when it is desired to apply the second level of electrosurgical energy to the second electrically conductive member 210, the electrosurgical energy source 20 can be set to a second mode of operation. According to other embodiments, the electrosurgical energy source 20 may be configured to concurrently deliver the first and second levels of electrosurgical energy to the electrosurgical instrument 10 so that the first and second levels of electrosurgical energy can be respectively applied to the first and second electrically conductive members 208, 210 concurrently. According to yet other embodiments, the level of the electrosurgical energy applied to the first electrically conductive member 208 and the level of electrosurgical energy applied to the second electrically conductive member 210 can be automatically selected by the electrosurgical surgical instrument 10 based on methodologies (e.g., the position of the first and second jaw members 202, 204) described in more detail hereinbelow. As the distance between the first and third electrically conductive members 208, 218 is less than the distance between the second and fourth electrically conductive members 210, 220, it will be appreciated that the compressive force applied by the first and third electrically conductive members 208, 218 to the portion of the tissue 206 positioned between the first and third electrically conductive members 208, 218 is greater than the compressive force applied by the second and fourth electrically conductive members 210, 220 to the portion of the tissue 206 positioned between the second and fourth electrically conductive members 210, 220. This is the case regardless of whether the end effector 200 is in the fully closed position or between the open and fully closed positions.

In view of the above, it will be appreciated that the electrosurgical surgical instrument 10 (or any other suitable surgical instrument) may be utilized to seal the tissue 206 positioned between the first and second jaw members 202, 204. By applying a lower compressive force and in some instances a lower level of electrosurgical energy to the portion of the tissue 206 that is positioned between the second and fourth electrically conductive members 210, 220 (as opposed to the high compressive force and in some instances a higher level of electrosurgical energy applied to the portion of the tissue 206 that is positioned between the first and third electrically conductive members 208, 218), the overall strength of the resulting seal of the tissue 206 is enhanced.

According to various embodiments, the first jaw member 202 may comprise a single electrically conductive member in lieu of the first and second electrically conductive members 208, 210. For such embodiments, the single electrically conductive member can have substantially the same shape as a combination of the first and second electrically conductive members 208, 210 such that in a plane which is transverse to the longitudinal axis 18, a minimum distance between a first portion of the single electrically conductive (corresponding to the first electrically conductive member 208) and the third electrically conductive member 218 is less than a minimum distance between a second portion of the single electrically conductive member (corresponding to the second electrically conductive member 210) and the fourth electrically conductive member 220. For such embodiments, the electrically insulative member 212 can be eliminated and the operation of the surgical instrument 10 is similar to that described hereinabove. For example, the compressive force applied by the first portion of the single electrically conductive member and the third electrically conductive member 218 to the portion of the tissue 206 positioned between the first portion of the single electrically conductive member and the third electrically conductive member 218 is greater than the compressive force applied by the second portion of the single electrically conductive member and the fourth electrically conductive member 220 to the portion of the tissue 206 positioned between the second portion of the single electrically conductive member and the fourth electrically conductive member 220. This is the case regardless of whether the end effector 200 is in the fully closed position or between the open and fully closed positions. Also, depending on which electrically conductive member electrosurgical energy is applied to, high-frequency current may be induced to flow, for example, (1) from the single electrically conductive member through the tissue to the third electrically conductive member 218, (2) from the single electrically conductive member through the tissue to the fourth electrically conductive member 220 or (3) from the third electrically conductive member 218 through the tissue to the fourth electrically conductive member 220. Of course, since the induced high frequency current is alternating current, it will be appreciated that the induced high frequency current is also induced to flow in a path which is “reverse” to that described hereinabove.

Similarly, according to other embodiments, the second jaw member 204 may comprise a single electrically conductive member in lieu of the third and fourth electrically conductive members 218, 220. For such embodiments, the single electrically conductive member of the second jaw member 204 can have substantially the same shape as a combination of the third and fourth electrically conductive members 218, 220 such that in a plane which is transverse to the longitudinal axis 18, a minimum distance between the first electrically conductive member 208 and a first portion of the single electrically conductive (corresponding to the third electrically conductive member 218) is less than a minimum distance between the second electrically conductive member 210 and a second portion of the single electrically conductive member (corresponding to the fourth electrically conductive member 220). For such embodiments, the electrically insulative member 222 can be eliminated and the operation of the surgical instrument 10 is similar to that described hereinabove. For example, the compressive force applied by the first electrically conductive member 208 and the first portion of the single electrically conductive member of the second jaw member 204 to the portion of the tissue 206 positioned between the first electrically conductive member 208 and the first portion of the single electrically conductive member is greater than the compressive force applied by the second electrically conductive member 210 and the second portion of the single electrically conductive member of the second jaw member 204 to the portion of the tissue 206 positioned between the second electrically conductive member 210 and the second portion of the single electrically conductive member. This is the case regardless of whether the end effector 200 is in the fully closed position or between the open and fully closed positions. Also, depending on which electrically conductive member electrosurgical energy is applied to, current may be induced to flow, for example, (1) from the first electrically conductive member 208 through the tissue to the first portion of the single electrically conductive member of the second jaw member 204, (2) from the second electrically conductive member 210 through the tissue to the second portion of the single electrically conductive member or (3) from the first electrically conductive member 208 through the tissue to the second electrically conductive member 210. Of course, since the induced high frequency current is alternating current, it will be appreciated that the induced high frequency current is also induced to flow in a path which is “reverse” to that described hereinabove.

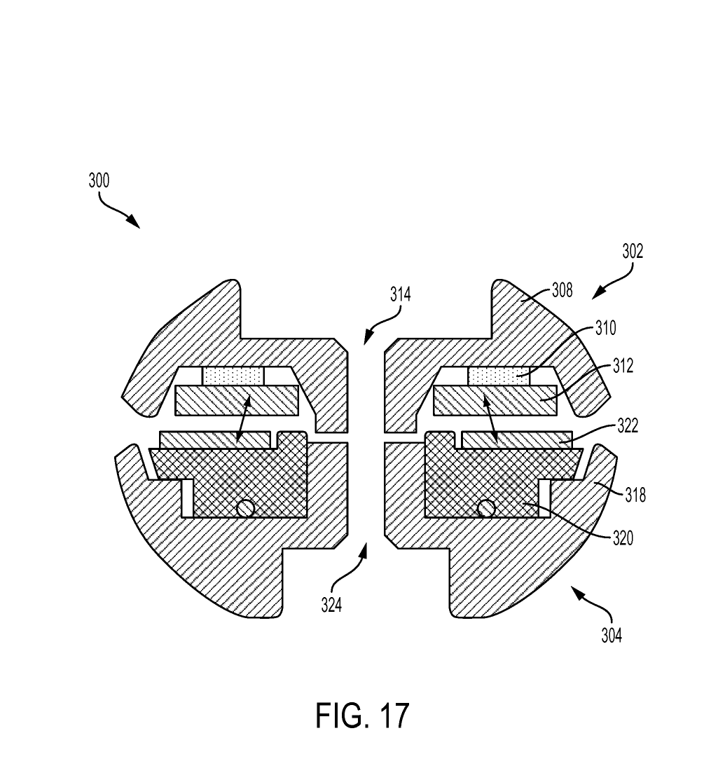

FIGS. 17-22 illustrate various embodiments of an end effector 300. The end effector 300 may be used in lieu of the end effector 16 or the end effector 200 with the electrosurgical instrument 10, and may also be used with any other any suitable electrosurgical instrument. Thus, according to various embodiments, the electrosurgical instrument 10 or any other suitable electrosurgical instrument may include the end effector 300. Although the end effector 300 shown in FIGS. 17-22 is axially aligned with the longitudinal axis 18, according to other embodiments the end effector 300 may be curved and the majority of the end effector 300 may not be axially aligned with the longitudinal axis 18. Except for certain differences noted hereinbelow, the end effector 300 is similar to the end effector 16 and the end effector 200.

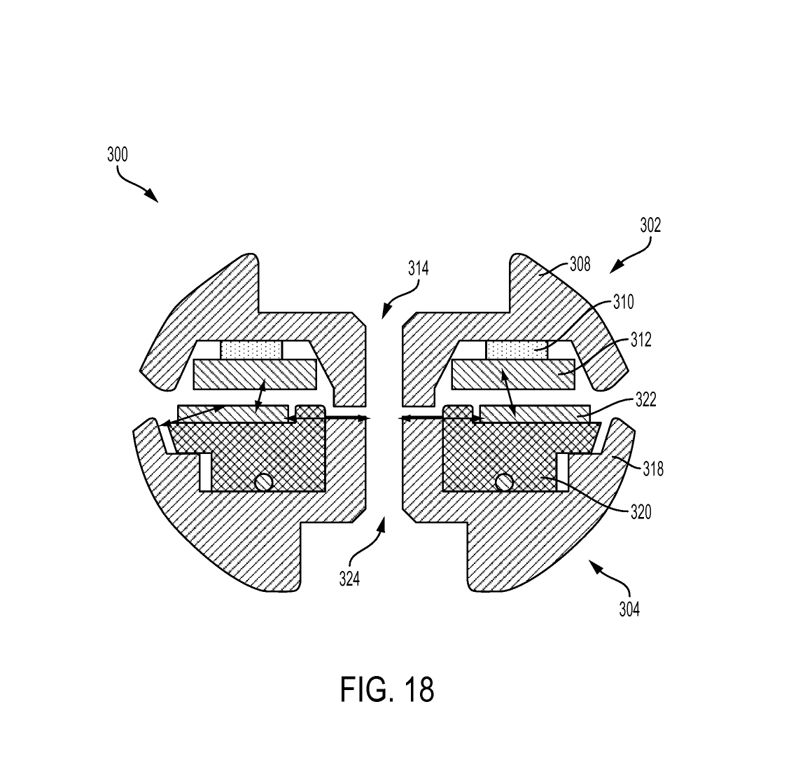

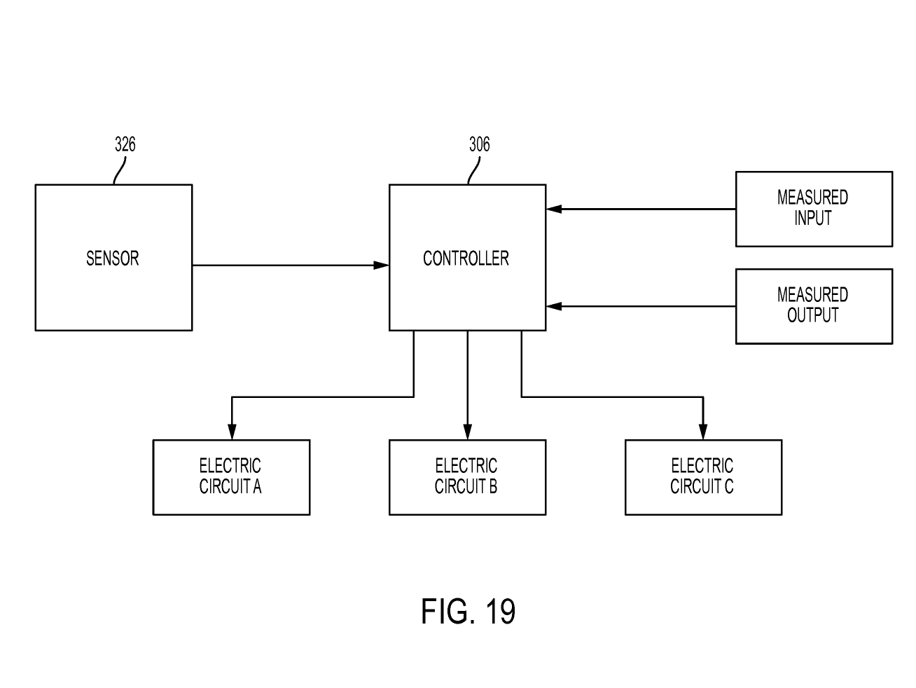

FIGS. 17-18 illustrate side views of an end effector 300 according to various embodiments. The end effector 300 includes a first jaw member 302 and a second jaw member 304. As explained in more detail hereinbelow, the end effector 300 may include a plurality of different electric circuits (See FIG. 19) which can be selectively coupled to the electrosurgical energy source 20 by a controller 306 (see FIG. 19) positioned within or external to the electrosurgical instrument 10 to deliver electrosurgical energy to a tissue positioned between the first and second jaw members 302, 304. Current paths between various components of the end effector 300 are shown by arrows in FIGS. 17-18.

At least one of the first and second jaw members 302, 304 are movable relative to one another in a manner identical or similar to that of the first and second jaw members 42, 44 described hereinabove. In some embodiments, the first and second jaw members 302, 304 are each movable relative to the other. In other embodiments, first jaw member 302 is in a fixed position and the second jaw member 304 is movable relative to the first jaw member 302. In yet other embodiments, the second jaw member 304 is in a fixed position and the first jaw member 302 is movable relative to the second jaw member 304. Regardless of the arrangement of the first and second jaw members 302, 304, the end effector 300 is movable between a first position and a second position. In the first position, which may be considered an open position, the first and second jaw members 302, 304 are spaced apart a maximum distance at their distal ends. In the first position, the end effector 300 is able to receive a tissue (not shown) such as, for example, a vessel, between the first and second jaw members 302, 304. In the second position, which may be considered a fully closed position, the first and second jaw members 302, 304 are spaced apart a minimum distance at their distal ends. As the end effector 300 is moved from the open position toward the fully closed position, the end effector 300 is able to apply compressive forces to the received tissue and deliver electrosurgical energy to the tissue. The electrosurgical energy delivered to the tissue may be, for example, RF energy, sub-therapeutic RF energy, ultrasonic energy and/or other suitable forms of energy. According to various embodiments, each of the first and second jaw members 302, 304 is adapted to connect to an opposite potential of the electrosurgical energy source 20 such that upon activation, the jaw members 302, 304 conduct bipolar energy through the tissue positioned between the jaw members 302, 304 to effect the tissue seal.

The first jaw member 302 includes a jaw body member 308, an electrically insulative member 310 and an electrically conductive member 312 (e.g., an electrode configured to deliver electrosurgical energy to tissue positioned between the first and second jaw members 302, 304). Additionally, according to various embodiments, the first jaw member 302 defines a slot or channel 314, and the channel 314 may be axially aligned with the longitudinal axis 18.

The jaw body member 308 may include any suitable conductive or non-conductive material. For embodiments where the jaw body member 308 is electrically conductive, the electrically conductive member 312 is electrically isolated from the jaw body member 308 by the electrically insulative member 310. Although not shown for purposes of simplicity in FIGS. 17-18, it will be appreciated that the electrically conductive member 312 is electrically coupled to the electrosurgical energy source 20 as explained in more detail hereinbelow. The electrically conductive member 312 may include any suitable electrically conductive material. For example, according to various embodiments, the electrically conductive member 312 may include an electrically conductive metal, an electrically conductive alloy, an electrically conductive positive temperature coefficient (PTC) material, an electrically conductive polymer, combinations thereof, etc. The electrically conductive material in the electrically conductive member 312 may be the same as or different than the electrically conductive material in the electrically conductive members 208, 210, 218, 220.

The electrically insulative member 310 is positioned between to the jaw body member 308 and the electrically conductive member 312. According to various embodiments, the electrically insulative member 310 is connected to the electrically conductive member 312 and/or the jaw body member 308. The electrically insulative member 310 may include any suitable type of electrically insulative material. For example, the electrically insulative material in the electrically insulative member 310 may be the same as or different than the electrically insulative material in the electrically insulative members 212, 222.

The second jaw member 304 includes a jaw body member 318, an electrically insulative member 320 and an electrically conductive member 322 (e.g., an electrode). Additionally, according to various embodiments, the second jaw member 304 defines a slot or channel 324, and the channel 324 may be axially aligned with the longitudinal axis 18.

The jaw body member 318 may include any suitable electrically conductive material, and the electrically conductive material may be the same as or different than the electrically conductive material in the jaw body member 308. The electrically insulative member 320 is positioned between the jaw body member 318 and the electrically conductive member 322. According to various embodiments, the electrically insulative member 320 is connected to the electrically conductive member 322 and/or the jaw body member 318. The electrically insulative member 320 may include any suitable electrically insulative material. For example, the electrically insulative material in the electrically insulative member 320 may be the same as or different than the electrically insulative material in the electrically insulative member 310.

The electrically conductive member 322 is electrically isolated from the jaw body member 318 by the electrically insulative member 320. Although not shown for purposes of simplicity in FIGS. 17-18, it will be appreciated that the electrically conductive member 322 is electrically coupled to the electrosurgical energy source 20. The electrically conductive member 322 may include any suitable electrically conductive material. The electrically conductive material in the electrically conductive member 322 may be the same as or different than the electrically conductive material in the electrically conductive member 312.

For embodiments of the end effector 300 which include the channels 314, 324, the channels 314, 324 cooperate to provide a path for an I-beam member or a C-beam member (neither of which are shown for purposes of simplicity) to advance from a position proximate the proximal end of the end effector 300 to a position proximate the distal end of the end effector 300. The I-beam member may be identical or similar to the I-beam member 158, the C-beam member may be identical or similar to the C-beam member described hereinabove, and either may be advanced distally in a manner the same as or different from that described hereinabove. In general, as the I-beam shaped member advances from a position proximate the proximal end of the end effector 300 to a position proximate the distal end of the end effector 300, the I-beam shaped member compresses (or further compresses) the portions of the tissue positioned between the first and second jaw members 302, 304 and may also cut through the tissue. For embodiments which do not include the channels 314, 324 and the I-beam or C-beam member, the surgical instrument utilized with the end effector 300 may include other types of channels (e.g., a knife channel), cutting and/or closure systems.

Additionally, although not shown in FIGS. 17-18 for purposes of simplicity, it will be appreciated that the electrosurgical instrument 10 (or other surgical instrument which includes the end effector 300) further includes a plurality of sensors 326 (See FIG. 19) which are within the electrosurgical instrument 10 (e.g., within the handle assembly 12, the end effector 300, etc.) and are coupled to the controller 306. The sensors 326 may be any suitable type of sensors. For example, according to various embodiments, the sensors 326 may be, for example, Hall-effect sensors, optical sensors, temperature sensors, pressure sensors, voltage sensors, current sensors, resistance sensors, combinations thereof, etc.

The controller 306 is configured to selectively couple different electric circuits to the electrosurgical energy source 20 to deliver electrosurgical energy to the tissue positioned between the first and second jaw members 302, 304. The controller 306 may be any suitable type of controller. For example, according to various embodiments the controller 306 is a switching device, a processor, an integrated circuit, etc. which is configured to selectively couple the appropriate electric circuit to the electrosurgical energy source 20. The electrosurgical instrument 10 may include any number of such electric circuits. For example, one such electric circuit includes the electrically conductive member 322, the tissue, and the electrically conductive member 312. For this electric circuit, the electrically conductive member 322 and the electrically conductive member 312 may be considered directly opposing one another. Another such electric circuit includes the electrically conductive member 322, the tissue and a cutting member (not shown) positioned in the channels 314, 324. For this electric circuit, the electrically conductive member 322 and the electrically conductive member 312 may be considered directly opposing one another, and the electrically conductive member 322 and the cutting member may be considered offset from one another. The cutting member may be embodied the same as or different than the cutting member 158. Yet another such electric circuit includes the electrically conductive member 322, the tissue, the jaw body member 318 and the electrically conductive member 312. For this electric circuit, the electrically conductive member 322 and the electrically conductive member 312 may be considered directly opposing one another, and the electrically conductive member 322 and the jaw body member 318 may be considered offset from one another. Other such electric circuits may include combinations of those described hereinabove.

The selective coupling of the appropriate electric circuit to the electrosurgical energy source 20 by the controller 306 may be realized in any number of different ways. For example, according to various embodiments, one or more sensors 326 located within the electrosurgical instrument 10 (e.g., within the handle assembly 12, the end effector 300, etc.) may be utilized to determine the position of the first and/or second jaw members 302, 304. The position of the first and/or second jaw members 302, 304 can be determined relative to a fully open position of the end effector 300, relative to a closed position of the end effector 300, relative to one another, etc. The outputs of the sensors 326 can be input to the controller 306 which is configured to (1) determine the position of the first and/or second jaw members 302, 304 based on the outputs of the sensors 326 and (2) based on the determined position of the first and/or second jaw members 302, 304, selectively couple an appropriate electric circuit to the electrosurgical energy source 20.

According to other embodiments, one or more of the sensors 326 may be utilized to determine the angle formed by the first and second jaw members 302, 304, and thus the relative positions of the first and second jaw members 302, 304. For such embodiments, the outputs of the sensors 326 can be input to the controller 306 which is configured to (1) determine the angle formed by the first and second jaw members 302, 304 based on the outputs of the sensors 326 and (2) based on the determined angle formed by the first and second jaw members 302, 304, selectively couple an appropriate electric circuit to the electrosurgical energy source 20.

According to yet other embodiments, the impedance of a tissue positioned between the first and second jaw members 302, 304 can be determined (e.g., by measuring certain characteristics of the tissue). For such embodiments, the measured characteristics can be input to the controller 306 to determine the impedance of the tissue, or the determined impedance can be input to the controller 306. Based on the determined impedance, the controller 306 is configured to selectively couple an appropriate electric circuit to the electrosurgical energy source 20.

According to yet other embodiments, various measured inputs and/or outputs can be utilized by the controller 306 to selectively couple an appropriate electric circuit to the electrosurgical energy source 20. Such measured inputs and/or outputs can be input to the controller 306 and may include, for example, the electrosurgical energy flowing into the tissue from the first or second jaw members 302, 304, the power flowing into the tissue from the first or second jaw members 302, 304, the current flowing into the tissue from the first or second jaw members 302, 304, the amount of time that energy, power and/or current has been flowing into the tissue from the first and/or second jaw members 302, 304, a temperature of the tissue as measured by one or more temperature sensors (e.g., sensor 326), a closure pressure being applied to the tissue as measured by one or more pressure sensors (e.g., sensor 326), etc.

In operation, when the end effector 300 is placed into the open position, a tissue can be positioned between the first and second jaw members 302, 304. As the end effector 300 is moved from the open position toward a closed position, the electrically conductive members 312, 322 eventually make contact with and cooperate to apply a compressive force to the portion of the tissue positioned between the electrically conductive members 312, 322. At this point, the controller 306 can selectively couple an appropriate electric circuit the based on (1) the position of the first and/or second jaw members 302, 304, (2) the angle formed by the first and second jaw members 302, 304, (3) the impedance of a tissue positioned between the first and second jaw members 302, 304 and/or (4) the above-described measured inputs and/or measured outputs.

For instances when the tissue positioned between the first and second jaw members 302, 304 is relatively “thick”, the electrosurgical instrument 10 can recognize that the tissue is relatively “thick” based on the determined position of the first and/or second jaw members 302, 304, or on the other methodologies (angle, impedance, measured inputs and/or measured outputs) described hereinabove. For such instances, the controller 306 can selectively couple the electric circuit which includes the electrically conductive member 322, the tissue and the electrically conductive member 312, for example, based on the determined position of the first and/or second jaw members 302, 304, and electrosurgical energy can be applied to that electric circuit. The electrosurgical energy can be applied to the electrically conductive member 322, and due to the difference in electrical potential between the electrically conductive members 312, 322, as well as the impedance associated with different current paths through the tissue, current is induced to flow (1) from the electrically conductive member 322 to the portion of the tissue positioned between the electrically conductive member 322 and the electrically conductive member 312, (2) through the portion of the tissue positioned between the electrically conductive member 322 and the electrically conductive member 312 and to the electrically conductive member 312 and (3) through the electrically conductive member 312. By utilizing the electric circuit which encourages this current path, the applied electrosurgical energy is utilized to cook down or debulk the portion of the tissue positioned between the electrically conductive member 322 and the electrically conductive member 312, thereby reducing the “thickness” the tissue. According to various embodiments, this electric circuit may be utilized when it is determined that the “thickness” of the tissue positioned between the electrically conductive member 322 and the electrically conductive member 312 is about 0.030 to 035 inches or greater. Of course, since the induced current is alternating current, it will be appreciated that the induced current is also induced to flow in a path which is “reverse” to that described hereinabove.

According to various embodiments, for instances where the “thickness” of the tissue positioned between the first and second jaw members 302, 304 is about 0.030-0.035 inches or less (either originally or after the debulking process is completed), the controller 306 can selectively couple the electric circuit which includes the electrically conductive member 322, the tissue, the jaw body member 318 and the electrically conductive member 312, for example, based on the determined position of the first and/or second jaw members 302, 304, and electrosurgical energy can be applied to that electric circuit. The electrosurgical energy can be applied to the electrically conductive member 322, and due to the difference in electrical potential between the electrically conductive member 322 and the jaw body member 318 and the electrically conductive member 312, as well as the impedance associated with different current paths through the tissue, current is induced to flow (1) from the electrically conductive member 322 to the portion of the tissue positioned between the electrically conductive member 322 and the jaw body member 318 and the electrically conductive member 312, (2) through the portion of the tissue positioned between the electrically conductive member 322 and the jaw body member 318 and the electrically conductive member 312 and (3) to the jaw body member 318 and the electrically conductive member 312. By utilizing the electric circuit which encourages this current path, the applied electrosurgical energy is utilized to seal the portion of the tissue positioned between the first and second jaw members 302, 304. Of course, since the induced current is alternating current, it will be appreciated that the induced current is also induced to flow in a path which is “reverse” to that described hereinabove.

According to other embodiments, for instances where the “thickness” of the tissue positioned between the first and second jaw members 302, 304 is about 0.030-0.035 inches or less (either originally or after the debulking process is completed), the controller 306 can selectively couple the electric circuit which includes the electrically conductive member 322, the tissue and the cutting member positioned in the channels 314, 324, for example, based on the determined position of the first and/or second jaw members 302, 304, and electrosurgical energy can be applied to that electrical circuit. The electrosurgical energy can be applied to the electrically conductive member 322, and due to the difference in electrical potential between the electrically conductive member 322 and the cutting member positioned in the channels 314, 324, as well as the impedance associated with different current paths through the tissue, current is induced to flow (1) from the electrically conductive member 322 to the portion of the tissue positioned between the electrically conductive member 322 and the cutting member, (2) through the portion of the tissue positioned between the electrically conductive member 322 and the cutting member and (3) to the cutting member. By utilizing the electric circuit which encourages this current path, the applied electrosurgical energy is utilized to seal and/or cut the portion of the tissue positioned between the first and second jaw members 302, 304. Of course, since the induced current is alternating current, it will be appreciated that the induced current is also induced to flow in a path which is “reverse” to that described hereinabove.

FIG. 19 is a simplified representation of electrical connections to the controller 306 according to various embodiments. Although only one sensor 326 is shown in FIG. 19, it will be appreciated that the electrosurgical instrument 10 may include any number of sensors 326. Similarly, although only one “measured input” connection and one “measured output” connection is shown as being an input to the controller 306, it will be appreciated that any number of measured inputs and/or measured outputs can be input to the controller 306. Also, although only three electric circuits are shown in FIG. 19 (electric circuit A, electric circuit B and electric circuit C), it will be appreciated that the electrosurgical instrument 10 may include any number of electric circuits which are configured to deliver electrosurgical energy to the tissue positioned between the first and second jaw members 302, 304.

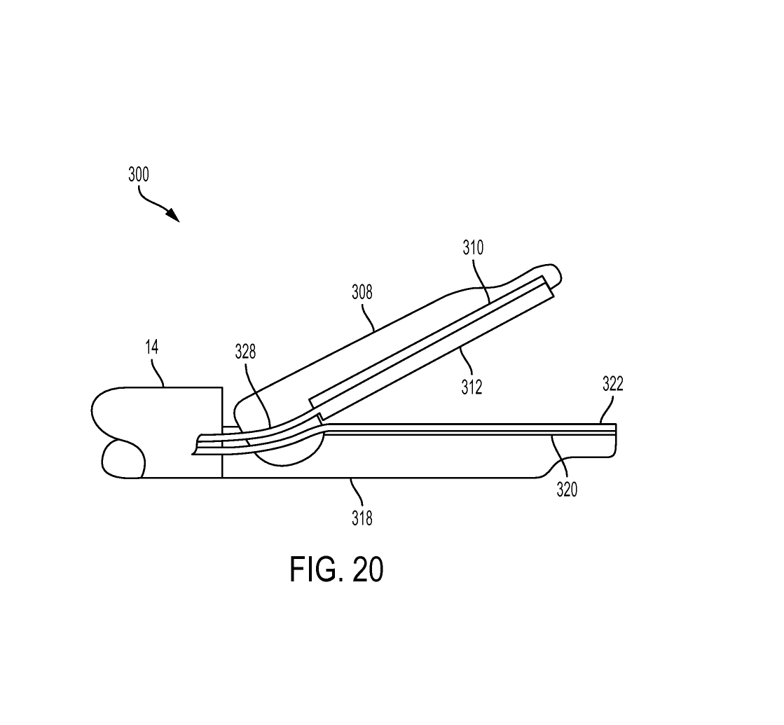

FIG. 20 illustrates other embodiments of the end effector 300. The embodiments shown in FIG. 20 are similar to those shown in FIGS. 17-18. The proximal ends of the respective electrically conductive members 312, 322 are shown as being electrically coupled to electrical conductors 328 (e.g., flexible stranded insulated wires) which may be the same as or different than the electrically conductive member/cables 98 described hereinabove. Although only two electrical conductors 328 are shown in FIG. 20, it will be appreciated that the electrosurgical instrument 10 may include any number of such electrical conductors 328 (e.g., one for each electrically conductive member, one for a ground, etc.). The electrical conductors 328 may extend into the shaft assembly 14 of the electrosurgical instrument 10 and provide a return path for the above-described electrical circuits. Thus, the above-described electrical circuits may include two or more of the electrical conductors 328.

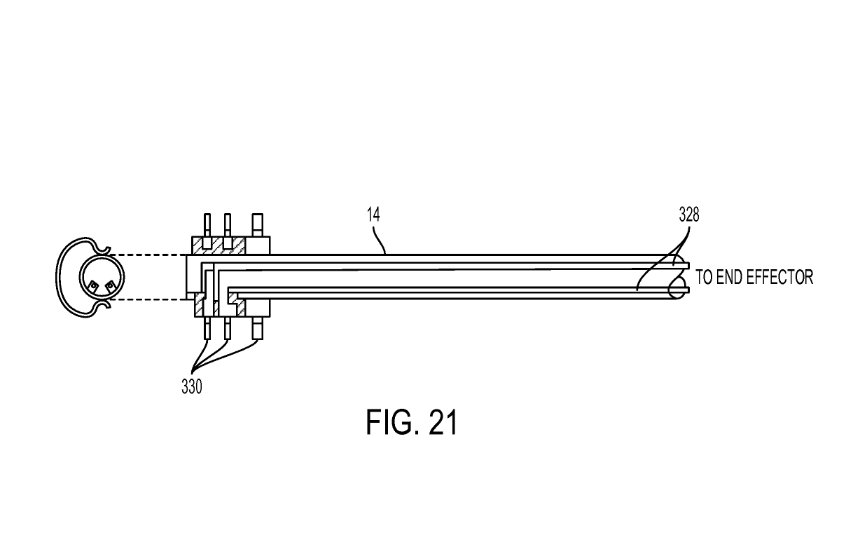

FIG. 21 illustrates connections of the respective proximal ends of the electrical conductors 328 according to various embodiments. As shown in FIG. 21, the electrosurgical instrument 10 includes the electrical conductors 328 which extend from the end effector 300 to the shaft assembly 14, and also includes a plurality of slip rings 330. The proximal ends of the electrical conductors 328 are coupled to the slip rings 330. The slip rings 330 operate to allow the transmission of electrosurgical energy from the handle assembly 12 (a stationary structure) to the shaft assembly 14 and the end effector 300 (rotating structures). As the shaft assembly 14, and by extension the end effector 300, rotate relative to the handle assembly 14, the slip rings 330 allow electrosurgical energy to be transferred from the electrically conductive cables 68 in the handle assembly 14 to the electrical conductors 328 in the shaft assembly 14. Each electrical conductor 328 has a corresponding electrically conductive cable 68 which it is coupled to via a corresponding slip ring 330. According to various embodiments, each slip ring 330 can include a circular, or an at least semi-circular, contact, for example, positioned within the handle assembly 12 which can remain in contact with a corresponding circular, or an at least semi-circular, contact positioned within the shaft assembly 14. Such corresponding contacts can permit relative rotational movement between the handle assembly 12 and the shaft assembly 114 and yet still provide an electrical path between the electrically conductive cables 68 and the electrical conductors 328. Although only three slip rings 330 are shown in FIG. 21, it will be appreciated that the electrosurgical instrument 10 may include any number of such slip rings 330. Furthermore, according to various embodiments, means other than the slip rings 330 may be utilized to permit relative rotational movement between the handle assembly 12 and the shaft assembly 114 and yet still provide an electrical path between the electrically conductive cables 68 and the electrical conductors 328 in the shaft assembly 14. Such means may include, for example, stereo jacks, pogo pins or other suitable means.

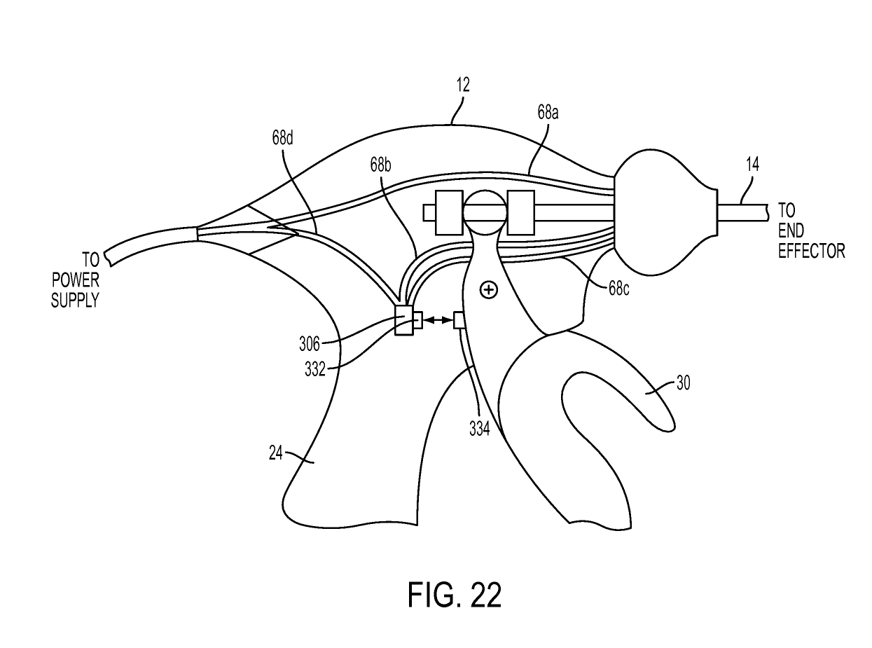

FIG. 22 illustrates a simplified representation of various embodiments of the handle assembly 12 which can be utilized with the end effector 300. As shown in FIG. 22, the controller 306 is positioned within the handle assembly 12 and is configured to selectively couple different electrically conductive cables 68 (e.g. flexible stranded insulated wires), which are associated with different electric circuits, to the electrosurgical energy source 20. For the embodiments shown in FIG. 22, the electrically conductive cable 68a is associated with the electrically conductive member 322 of the second jaw member 304, the electrically conductive cable 68b is associated with the electrically conductive member 312 of the first jaw member 302 and the electrically conductive cable 68c is associated with the jaw body member 318. The electrically conductive cable 68d operates as the return to the electrosurgical energy source 20. Although not shown in FIG. 22, it will be appreciated that the handle assembly 12 may also include an electrically conductive cable (not shown) associated with the cutting member, as well as any number of other electrically conductive cables 68 which are associated with other electric circuits and which serve as return current paths.

For the embodiments shown in FIG. 22, the controller 306 is implemented as a switching device. For these embodiments, when the position of the trigger 30 relative to the pistol grip 24 is indicative of the tissue positioned between the first and second jaw members 302, 304 having a “thickness” of about 0.030-0.035 inches or greater, the controller 306 selectively couples the electric circuit which includes the electrically conductive member 322, the tissue and the electrically conductive member 312 to the electrosurgical energy source 20. When the energy button 32 is then depressed, electrosurgical energy is delivered to the coupled electric circuit. When the trigger 30 reaches a position relative to the pistol grip 24 which is indicative of the tissue having a “thickness” which is less than about 0.030-0.035 inches, a switch/push button 332 of the controller 306 is depressed by a protuberance 334 on the trigger 30, and the depressed switch/push button 332 operates to selectively couple the electric circuit which includes the electrically conductive member 322, the tissue, the jaw body member 318 and the electrically conductive member 312 to the electrosurgical energy source 20. When the energy button 32 is then depressed, energy is delivered to the coupled electric circuit. Alternatively, the depressed switch/push button 332 can operate to selectively couple the electric circuit which includes the electrically conductive member 322, the tissue and the cutting member to the electrosurgical energy source 20. Of course, according to different embodiments of the controller 306, the selective coupling of the electric circuits to the electrosurgical energy source 20 may be realized in different ways.

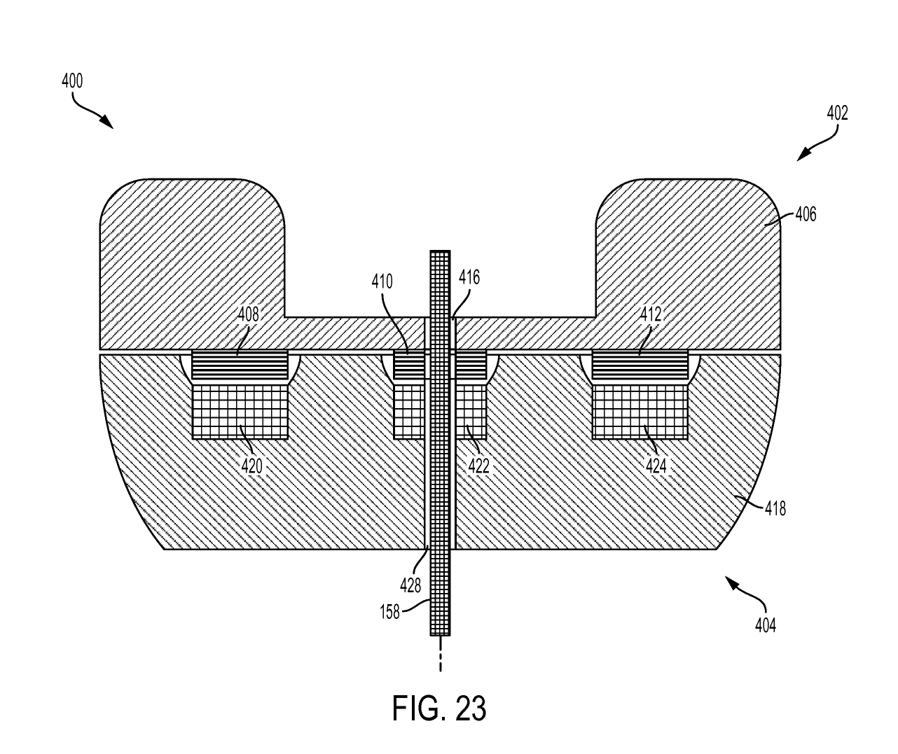

FIGS. 23-25 illustrate various embodiments of an end effector 400. The end effector 400 may be used in lieu of the end effector 16, the end effector 200 or the end effector 300 with the electrosurgical instrument 10, and may also be used with any other any suitable electrosurgical instrument. Thus, according to various embodiments, the electrosurgical instrument 10 or any other suitable electrosurgical instrument may include the end effector 400. Although the end effector 400 shown in FIGS. 23-25 is axially aligned with the longitudinal axis 18, according to other embodiments the end effector 400 may be curved and the majority of the end effector 400 may not be axially aligned with the longitudinal axis 18. Except for certain differences noted hereinbelow, the end effector 400 is similar to the end effector 16, the end effector 200 and the end effector 300.

FIG. 23 is a front view of the end effector 400 according to various embodiments. The end effector 400 includes a first jaw member 402 and a second jaw member 404. As explained in more detail hereinbelow, the end effector 400 may include a plurality of different electric circuits which can be selectively coupled to the electrosurgical energy source 20 by the controller 306 to deliver electrosurgical energy to a tissue (e.g., a vessel) positioned between the first and second jaw members 402, 404, wherein at least one of the electric circuits includes two electrically conductive members (e.g., electrodes) from one of the first or second jaw members 402, 404 and one electrically conductive member (e.g., an electrode) from the other of the first and second jaw members 402, 404.

At least one of the first and second jaw members 402, 404 are movable relative to one another in a manner identical or similar to that of the first and second jaw members 42, 44 described hereinabove. In some embodiments, the first and second jaw members 402, 404 are each movable relative to the other. In other embodiments, first jaw member 402 is in a fixed position and the second jaw member 404 is movable relative to the first jaw member 402. In yet other embodiments, the second jaw member 404 is in a fixed position and the first jaw member 402 is movable relative to the second jaw member 404. Regardless of the arrangement of the first and second jaw members 402, 404, the end effector 400 is movable between a first position and a second position. In the first position, which may be considered an open position, the first and second jaw members 402, 404 are spaced apart a maximum distance at their distal ends. In the first position, the end effector 400 is able to receive a tissue (not shown) between the first and second jaw members 402, 404. In the second position, which may be considered a fully closed position, the first and second jaw members 402, 404 are spaced apart a minimum distance at their distal ends. As the end effector 400 is moved from the open position toward the fully closed position, the end effector 400 is able to apply compressive forces to the received tissue and deliver electrosurgical energy to the tissue. The electrosurgical energy delivered to the tissue may be, for example, RF energy, sub-therapeutic RF energy, ultrasonic energy and/or other suitable forms of energy. According to various embodiments, each of the first and second jaw members 402, 404 is adapted to connect to an opposite potential of the electrosurgical energy source 20 such that upon activation, the jaw members 402, 404 conduct bipolar energy through the tissue positioned between the jaw members 402, 404 to effect the tissue seal.

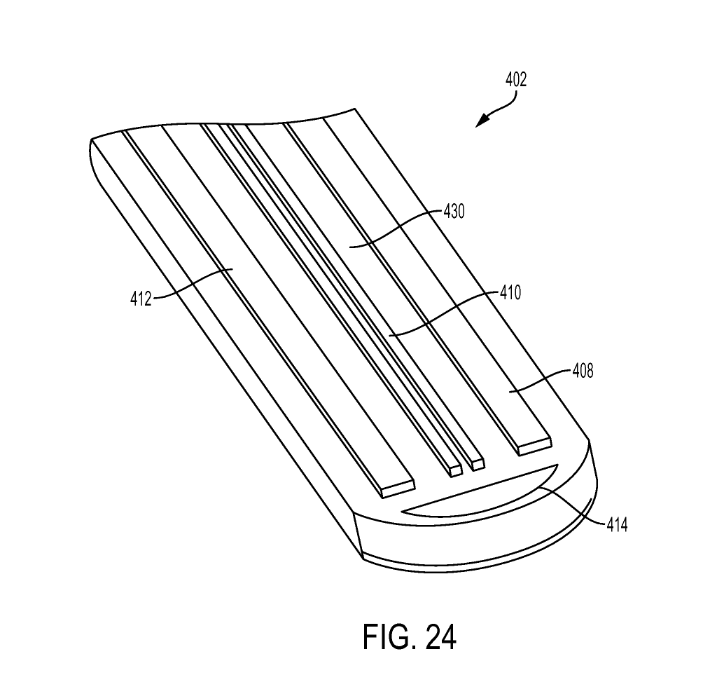

The first jaw member 402 includes a jaw body member 406, a first electrically conductive member 408 (e.g., an electrode configured to deliver electrosurgical energy to the tissue positioned between the first and second jaw members 402, 404), a second electrically conductive electrode 410 (e.g., an electrode) and a third electrically conductive member 412 (e.g., an electrode). Although not shown in FIG. 23, the first jaw member 402 may also includes a fourth electrically conductive member 414 (e.g., an electrode) as shown, for example, in FIG. 24. Additionally, according to various embodiments, the first jaw member 404 defines a slot or channel 416, and the channel 416 may be axially aligned with the longitudinal axis 18.

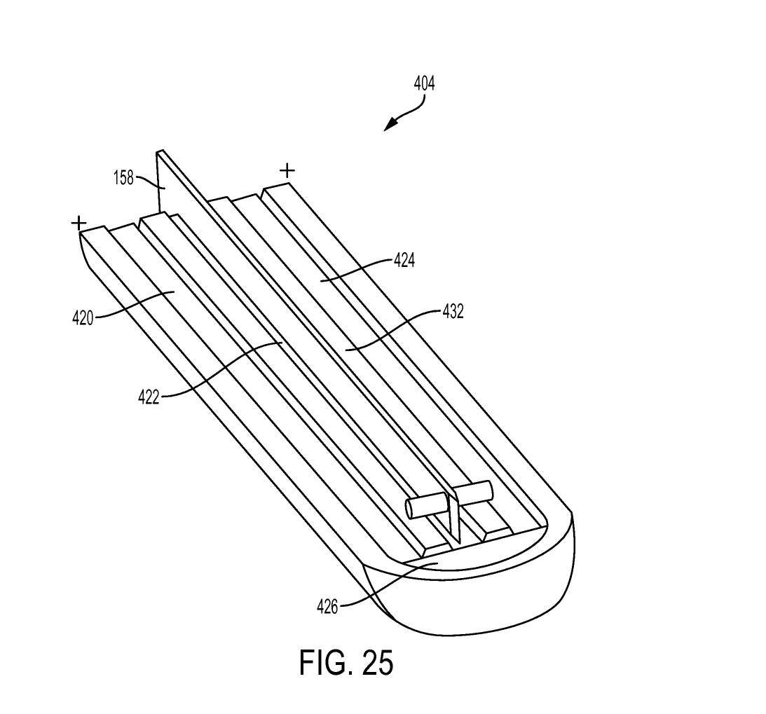

Similarly, the second jaw member 404 includes a jaw body member 418, a first electrically conductive member 420 (e.g., an electrode), a second electrically conductive electrode 422 (e.g., an electrode) and a third electrically conductive member 424 (e.g., an electrode). Although not shown in FIG. 23, the second jaw member 404 may also include a fourth electrically conductive member 426 (e.g., an electrode) as shown, for example, in FIG. 25. Additionally, according to various embodiments, the second jaw member 404 defines a slot or channel 428, and the channel 428 may be axially aligned with the longitudinal axis. Although the first electrically conductive member 408 is shown as being axially aligned with the first conductive electrically member 420, and the third electrically conductive member 412 is shown as being axially aligned with the third electrically conductive member 424, it will be appreciated that according to other embodiments the first electrically conductive members 408, 420 are not axially aligned with one another and/or the third electrically conductive members 412, 424 are not axially aligned with one another. In other words, for such embodiments, the first electrically conductive members 408, 420 are offset from one another and/or the third electrically conductive members 412, 424 are offset from one another.

For embodiments of the end effector 400 which include the channels 416, 428, the respective second electrically conductive members 410, 422 may be split into two portions (e.g., one-half on the “left” of the cutting member and one-half on the “right” side of the cutting member), and the channels 416, 428 cooperate to provide a path for an I-beam member or a C-beam member to advance from a position proximate the proximal end of the end effector 400 to a position proximate the distal end of the end effector 400. The I-beam member may be identical or similar to the I-beam member 158, the C-beam member may be identical or similar to the C-beam member described hereinabove, and either may be advanced distally in a manner the same as or different from that described hereinabove. In general, as the I-beam shaped member advances from a position proximate the proximal end of the end effector 400 to a position proximate the distal end of the end effector 400, the I-beam shaped member compresses (or further compresses) the portions of the tissue positioned between the first and second jaw members 402, 404 and may also cut through the tissue. For embodiments which do not include the channels 416, 428 and the I-beam or C-beam member, the electrosurgical instrument 10 utilized with the end effector 400 may include other types of channels (e.g., a knife channel), cutting and/or closure systems.

The controller 306 is configured to selectively couple different electric circuits to the electrosurgical energy source 20 to deliver electrosurgical energy to the tissue positioned between the first and second jaw members 402, 404. The electrosurgical instrument 10 may include any number of such electric circuits. For example, one such electric circuit includes the first electrically conductive member 408, the tissue, the second electrically conductive member 422 and the third electrically conductive member 412. A similar electric circuit includes the first electrically conductive member 420, the tissue, the second electrically conductive member 410 and the third electrically conductive member 424. Another electric circuit includes the first electrically conductive member 408, the tissue and the first electrically conductive member 420. Another electric circuit includes the first electrically conductive member 408, the tissue and the first electrically conductive member 420. A similar electric circuit includes the second electrically conductive member 410, the tissue and the second electrically conductive member 422. Another similar circuit includes the third electrically conductive member 412, the tissue and the third electrically conductive member 424. Yet another similar circuit includes the fourth electrically conductive member 414, the tissue and the fourth electrically conductive member 426. Other such electric circuits may include combinations of those described hereinabove.

In operation, when the end effector 400 is placed into the open position, a tissue can be positioned between the first and second jaw members 402, 404. As the end effector 400 is moved from the open position toward a closed position, the electrically conductive members of the first and second jaw members 402, 404 eventually make contact with and cooperate to apply a compressive force to the portion of the tissue positioned between the first and second jaw members 402, 404. At this point, the controller 306 can selectively couple an appropriate electric circuit the based on (1) the position of the first and/or second jaw members 402, 404, (2) the angle formed by the first and second jaw members 402, 404, (3) the impedance of a tissue positioned between the first and second jaw members 402, 404, (4) the above-described measured inputs and/or measured outputs and/or (5) a selected mode of operation.

According to various embodiments, at least four different modes of operation may be selected, and the selected mode determines the electric circuit (or electric circuits) which are selectively coupled to the electrosurgical energy source 20. The modes may be selected by a user or from the methodologies (e.g., position of jaw members, angle, impedance, measured inputs and/or measured outputs) as described hereinabove. A first mode of operation may be considered a “cook down” mode, a second mode of operation may be considered a “seal mode, a third mode of operation may be considered a “cutting” mode and a fourth mode of operation may be considered a “touch up” mode.

For instances where the “cook down” mode is selected, the electric circuit which includes the second electrically conductive member 410, the tissue and the second electrically conductive member 422 is selectively coupled to the electrosurgical energy source 20. In the “cook down” mode, the current density in the tissue is relatively high. Similarly, for instances where the “cutting” mode is selected, the electric circuit which includes the second electrically conductive member 410, the tissue and the second electrically conductive member 422 is selectively coupled to the electrosurgical energy source 20. For instances where the “touch up” mode is selected, the electric circuit which includes the fourth electrically conductive member 414, the tissue and the fourth electrically conductive member 426 is selectively coupled to the electrosurgical energy source 20, and the current density in the tissue is relatively low. Each of the current paths resulting from the “cook down” mode, the “cutting” mode and the “touch up” mode can be considered a direct current path.

For instances where the “seal” mode is selected, one of three different electric circuits can be selectively coupled to the electrosurgical energy source 20. In some embodiments, the electric circuit which includes the first electrically conductive member 408, the tissue, the second electrically conductive member 422 and the third electrically conductive member 412 is selectively coupled to the electrosurgical energy source 20 when the “seal” mode is selected. For these embodiments, the current path resulting from this mode of operation may be considered an indirect or offset current path. In other embodiments, the electric circuit which includes the first electrically conductive member 420, the tissue, the second electrically conductive member 410 and the third electrically conductive member 424 is selectively coupled to the electrosurgical energy source 20 when the “seal” mode is selected. For these embodiments, the current path resulting from this mode of operation may be considered an indirect or offset current path. Additionally, according to various embodiments, the selective coupling to the electrosurgical energy source 20 may toggle between the electric circuit which includes the first electrically conductive member 408, the tissue, the second electrically conductive member 422 and the third electrically conductive member 412 and the electric circuit which includes the first electrically conductive member 420, the tissue, the second electrically conductive member 410 and the third electrically conductive member 424 when the “seal” mode is selected.

In yet other embodiments, the following two circuits are concurrently selectively coupled to the electrosurgical energy source 20 when the “seal” mode is selected: (1) the electric circuit which includes the first electrically conductive member 408, the tissue and the first electrically conductive member 420 and (2) the electric circuit which includes the third electrically conductive member 412, the tissue and the third electrically conductive member 412. For these embodiments, each of the current paths resulting from this mode of operation may be considered direct current paths. In view of the above, it will be appreciated that each of the electrically conductive members 408-414 and 420-426 can be independently controlled. In other words, each of the electrically conductive members 408-414 and 420-426 are individually addressable.

According to various embodiments, the controller 306 can change, actuate or automatically set the mode of operation when a determined impedance reaches a level indicative of tissue being present between the first and second jaw members 402, 404. For example, when tissue is present between the distal ends of the first and second jaw members 402, 404, the fourth electrically conductive members 414, 426 may be utilized to determine the impedance of the tissue therebetween. Based on the determined impedance, the controller 306 can actuate the “touch up” mode by selectively coupling the fourth electrically conductive members 414, 426 to the electrosurgical energy source 20.

According to other embodiments, the controller 306 can control the various electrically conductive members (e.g., electrodes) to operate in a cycling mode, changing from one individual mode to another (e.g., “cook down” to “seal” to “cutting” to “touch up”) until each individual mode of operation has been utilized. According to various embodiments, the controller 306 can also control the various electrically conductive members to operate in a partial cycling mode, where less than all of the individual modes are utilized. In the cycling or partial cycling mode, for a given individual mode, the individual current path created by the coupling of the electrically conductive members can be utilized to independently monitor tissue impedance in the given mode. The cycling or partial cycling mode can be repeated any number of times (cycles) until a specified level of tissue impedance (a tissue impedance threshold) is realized for each individual mode. Once a target tissue impedance in a given mode has been realized, the cycle can skip that mode during subsequent cycles of the mode cycling process.

FIG. 24 is a perspective view of the bottom of the first jaw member 402 according to various embodiments. As shown in FIG. 24, the first, second and third electrically conductive members 408, 410, 412 extend longitudinally, are oriented parallel to one another, and are electrically isolated from one another by an electrically insulative member 430. The fourth electrically conductive member 414 is oriented transverse to the first, second and third electrically conductive members 408, 410, 412 and is electrically isolated from the first, second and third electrically conductive members 408, 410, 412 by the electrically insulative member 430.

FIG. 25 is a perspective view of the top of the second jaw member 404 according to various embodiments. As shown in FIG. 25, the first, second and third electrically conductive members 420, 422, 424 extend longitudinally, are oriented parallel to one another, and are electrically isolated from one another by an electrically insulative member 432. The fourth electrically conductive member 426 is oriented transverse to the first, second and third electrically conductive members 420, 422, 424 and is electrically isolated from the first, second and third conductive members 420, 422, 424 by the electrically insulative member 432. For the embodiments shown in FIGS. 24 and 25, it will be appreciated that the fourth electrically conductive member 414 and the fourth electrically conductive member 426 are transversely aligned with one another. According to other embodiments, the fourth electrically conductive members 414, 426 are not transversely aligned with one another. In other words, for such embodiments, the fourth electrically conductive members 414, 426 are offset from one another.

It is worthy to note that any reference to “one aspect,” “an aspect,” “one embodiment,” or “an embodiment” means that a particular feature, structure, or characteristic described in connection with the aspect is included in at least one aspect. Thus, appearances of the phrases “in one aspect,” “in an aspect,” “in one embodiment,” or “in an embodiment” in various places throughout the specification are not necessarily all referring to the same aspect. Furthermore, the particular features, structures or characteristics may be combined in any suitable manner in one or more aspects.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

What is claimed is:

1. An electrosurgical instrument, comprising:
an end effector movable between a first position and a second position, the end effector comprising:
a first jaw member comprising:
a first electrically conductive member; and
a second electrically conductive member electrically isolated from the first electrically conductive member; and
a second jaw member comprising a third electrically conductive member, wherein the first jaw member is movable relative to the second jaw member from an open position to a closed position to grasp a tissue positioned between the first and second jaw members, at least one of the first and second jaw members being adapted to connect to an electrosurgical energy source such that electrosurgical energy can be selectively communicated through the tissue positioned between the first and second jaw members to effect a tissue seal; and
wherein a distance between the first electrically conductive member and the third electrically conductive member is less than a distance between the second electrically conductive member and the third electrically conductive member.

2. The electrosurgical instrument of claim 1, wherein at least one of the first, second and third electrically conductive members is an electrode configured to deliver electrosurgical energy to the tissue positioned between the first and second jaw members.

3. The electrosurgical instrument of claim 1, wherein the electrosurgical instrument is configured to apply:
a first level of electrosurgical energy to the first electrically conductive member; and
a second level of electrosurgical energy to the second electrically conductive member.

4. The electrosurgical instrument of claim 3, wherein the first level of electrosurgical energy is greater than the second level of electrosurgical energy.

5. The electrosurgical instrument of claim 3, wherein the electrosurgical energy is one of the following:
a radio-frequency energy; and
a sub-therapeutic radio-frequency energy.

6. The electrosurgical instrument of claim 1, wherein:
the first and third electrically conductive members are configured to collectively apply a first closure pressure to the tissue positioned between the first and second jaw members; and
the second and third electrically conductive members are configured to collectively apply a second closure pressure to the tissue, wherein the first closure pressure is greater than the second closure pressure.

7. The electrosurgical instrument of claim 1, wherein the first jaw member further comprises an electrically insulative member positioned between the first and second electrically conductive members.

8. The electrosurgical instrument of claim 1, wherein the second jaw member further comprises a fourth electrically conductive member electrically isolated from the third electrically conductive member, wherein the distance between the first electrically conductive member and the third electrically conductive member is less than a distance between the second electrically conductive member and the fourth electrically conductive member.

9. The electrosurgical instrument of claim 8, further comprising:
a first electrically insulative member positioned between the first and second electrically conductive members; and
a second electrically insulative member positioned between the third and fourth electrically conducting members.

10. The electrosurgical instrument of claim 1, wherein each of the first and second jaw members is adapted to connect to an opposite potential of the electrosurgical energy source such that upon activation, the first and second jaw members conduct bipolar energy through the tissue positioned between the first and second jaw members to effect the tissue seal.

11. An electrosurgical instrument, comprising:
an end effector movable between a first position and a second position, the end effector comprising:
a first jaw member comprising:
a first jaw body member; and
a first electrically conductive member electrically isolated from the first jaw body member; and
a second jaw member comprising:
a second jaw body member; and
a second electrically conductive member electrically isolated from the jaw body member, wherein the first jaw member is movable relative to the second jaw member from an open position to a closed position to grasp a tissue positioned between the first and second jaw members, at least one of the first and second jaw members being adapted to connect to an electrosurgical energy source such that electrosurgical energy can be selectively communicated through the tissue positioned between the first and second jaw members to effect a tissue seal; and
a controller configured to selectively electrically couple:
the second electrically conductive member with the first electrically conductive member; and
the second electrically conductive member with the second jaw body member.

12. The electrosurgical instrument of claim 11, further comprising a cutting member, wherein the controller is further configured to selectively electrically couple the second electrically conductive member with the cutting member.

13. The electrosurgical instrument of claim 11, wherein the first electrically conductive member comprises a positive temperature coefficient material.

14. The electrosurgical instrument of claim 13, wherein the first jaw member further comprises an electrically insulative member positioned between the first jaw body member and the first electrically conductive member.

15. The electrosurgical instrument of claim 11, wherein the second jaw member further comprises an electrically insulative member positioned between the second jaw body member and the second electrically conductive member.

16. The electrosurgical instrument of claim 11, further comprising a sensor, wherein the sensor is electrically coupled to the controller.

17. The electrosurgical instrument of claim 11, further comprising a plurality of sensors electrically coupled to the controller.

18. The electrosurgical instrument of claim 11, wherein each of the first and second electrically conductive members is adapted to connect to an opposite potential of the electrosurgical energy source such that upon activation, the first and second electrically conductive members communicate bipolar energy through the tissue positioned between the first and second jaw members to effect the tissue seal.

19. The electrosurgical instrument of claim 11, wherein each of the first and second electrically conductive members is adapted to connect to an opposite potential of the electrosurgical energy source and the second jaw body member is connected to one potential of the electrosurgical energy source such that upon activation, the first and second electrically conductive members and the second jaw body member communicate bipolar energy through the tissue positioned between the first and second jaw members to effect the tissue seal.

20. The electrosurgical instrument of claim 11, wherein the second electrically conductive member is selectively electrically coupled with the first electrically conductive member and second jaw body member based on a position of at least one of the first and second jaw members.

21. An electrosurgical instrument, comprising:
an end effector movable between a first position and a second position, the end effector comprising:
a first jaw member comprising a first plurality of electrically conductive members, wherein each of the first plurality of electrically conductive members are electrically isolated from one another; and
a second jaw member comprising a second plurality of electrically conductive members, wherein each of the second plurality of electrically conductive members are electrically isolated from one another, wherein the first jaw member is movable relative to the second jaw member from an open position to a closed position to grasp a tissue positioned between the first and second jaw members, at least one of the first and second jaw members being adapted to connect to an electrosurgical energy source such that electrosurgical energy can be selectively communicated through the tissue positioned between the first and second jaw members to effect a tissue seal; and
a controller configured to selectively electrically couple two electrically conductive members of the first plurality of electrically conductive members with one electrically conductive member of the second plurality of electrically conductive members.

22. The electrosurgical instrument of claim 21, wherein:
each of the first plurality of electrically conductive members are individually addressable; and
each of the second plurality of electrically conductive members are individually addressable.

23. The electrosurgical instrument of claim 21, wherein at least one of the first plurality of electrically conductive members is offset from a corresponding electrically conductive member of the second plurality of electrically conductive members.

24. The electrosurgical instrument of claim 21, wherein the first plurality of conductive members comprises:
a first electrically conductive member;
a second electrically conductive member; and
a third electrically conductive member, wherein each of the first, second and third electrically conductive members extend longitudinally and are oriented parallel relative to each other.

25. The electrosurgical instrument of claim 24, wherein the first plurality of electrically conductive members further comprises a fourth electrically conductive member, wherein the fourth electrically conductive member is oriented transverse to the first, second, and third electrically conductive members.

26. The electrosurgical instrument of claim 24, wherein the second plurality of conductive members comprises:
a first electrically conductive member;
a second electrically conductive member; and
a third conductive electrically member, wherein each of the first, second and third electrically conductive members of the second plurality of electrically conductive members extend longitudinally and are oriented parallel relative to each other.

27. The electrosurgical instrument of claim 26, wherein the second plurality of electrically conductive members further comprises a fourth electrically conductive member, wherein the fourth electrically conductive member of the second plurality of electrically conductive members is oriented transverse to the first, second and third electrically conductive members of the second plurality of electrically conductive members.

28. The electrosurgical instrument of claim 21, wherein the controller is further configured to selectively electrically couple one electrically conductive member of the first jaw member with two electrically conductive members of the second jaw member.

29. The electrosurgical instrument of claim 21, wherein the controller is further configured to concurrently selectively electrically couple:
a first one of the first plurality of electrically conductive members with a corresponding first one of the second plurality of electrically conductive members; and
a second one of the first plurality of electrically conductive members with a corresponding second one of the second plurality of electrically conductive members.

30. The electrosurgical instrument of claim 21, wherein the controller is further configured to selectively couple one electrically conductive member of the first plurality of electrically conductive members with a corresponding electrically conductive member of the second plurality of electrically conductive members.

31. The electrosurgical instrument of claim 21, wherein the controller is further configured to cycle the electrosurgical instrument through a plurality of operating modes, wherein each respective operating mode is associated with a different combination of electrically coupled electrically conductive members of the first and second sets of electrically conductive members.
